(19) United States
(12) Patent Application Publication
(10) Pub. No.: US 2007/0259829 A1
(43) Pub. Date: Nov. 8, 2007

(54) Uses of diphenyl/diphenylamine carboxylic acids

(76) Inventors: Maen Abdelrahim, Orlando, FL (US); Stephen H. Safe, College Station, TX (US)

Correspondence Address: Benjamin Aaron Adler;ADLER & ASSOCIATES, 8011 Candle Lane, Houston, TX, 77071, US

(21) Appl. No.: 11/728,566

(22) Filed: Mar. 26, 2007

Related U.S. Application Data
(60) Provisional application No. 60/785,730, filed on Mar. 24, 2006.

(51) Int. Cl. A61K 31/192; A61K 31/7008; A61P 35/00; A61P 35/04; A61P 9/00; C12N 5/02

(52) U.S. Cl. 514/44; 435/375; 514/568

(57) ABSTRACT

The present invention demonstrates that chemical-induced degradation of Sp proteins by a specific sub-class of NSAIDs inhibited cancer cell growth, angiogenesis and metastasis of cancer cells. The inhibitory effects of these compounds were demonstrated in vitro and in vivo. Hence, the results discussed herein indicate that these compounds can be used to inhibit cell growth, angiogenesis and metastasis in cancers such as pancreatic, breast, prostate, colon, bladder and ovarian cancers.

### CROSS-REFERENCE TO RELATED APPLICATION

This non-provisional application claims benefit of provisional U.S. Ser. No. 60/785,730, filed Mar. 24, 2006, now abandoned.

### BACKGROUND OF THE INVENTION

1. Field of the Invention

The present invention relates to the fields of cell signaling pertaining to tumor cell growth, angiogenesis and metastasis. More specifically, the present invention discloses degradation of Sp family proteins by a specific sub-class of nonsteroidal antiinflammatory drugs (NSAIDs) and related compounds, which results in inhibition of growth, angiogenesis and metastasis of pancreatic cancer.

2. Description of the Related Art

Development of novel therapies for treating pancreatic cancer and other highly aggressive tumors requires a basic understanding of their critical growth regulatory and angiogenic pathways. Pancreatic carcinoma is the fourth leading cause of cancer mortality in the US, with more than 28,000 deaths attributed to this disease each year. Pancreatic cancer is associated with a death:incidence ratio of approximately 0.99. The incidence of pancreatic cancer in the US has increased nearly three-fold from 1920 to 1978. Pancreatic cancer is characterized by a high metastatic potential and rapid progression with a median survival rate of only 24 weeks in untreated cases. Due to local invasion and/or metastasis, only 15-20% of pancreatic cancer patients qualify for surgical intervention. For locally advanced, unresectable, and metastatic disease, treatment is palliative at best and usually consists of 5-fluorouracil or gemcitabine alone, or in combination with radiotherapy. Unfortunately, despite the moderate success of gemcitabine (2′,2′-difluorodeoxycitidine) median survival rates remain under 6 months for patients with metastatic disease. Given the poor performance of existing therapies, there is an increasing need to develop alternative drugs that target specific pathways that inhibit angiogenesis and tumor growth/regression.

Transcription factors are now recognized as targets for development of new anticancer drugs, and Sp-dependent gene expression is known to play critical roles in tumor development, growth and metastasis. Sp1 is overexpressed in pancreatic cancer compared to normal tissues and several studies have linked elevated Sp protein expression to upregulation of genes that are involved in pancreatic tumor growth and metastasis and these include p27 (suppressor) and vascular endothelial growth factor (VEGF) and its receptors. Sp proteins play a critical role in growth and metastasis of cancer (8-10), and there is evidence that Sp1 expression is a negative prognostic factor for survival in some cancer patients (11-16). These observations are not surprising since Sp1 and other Sp proteins that bind GC-rich promoter sites are transcription factors that regulate key sets of genes responsible for cancer cell proliferation and angiogenesis (8-11, 17-19).

Previous studies showed the Sp1 protein interactions with a proximal GC-rich motif in the VEGF was important for VEGF expression (11), and RNA interference was used to determine the role of Sp1, Sp3 and Sp4 in mediating expression of this important angiogenic factor (17). Using a series of constructs containing VEGF promoter inserts, it was initially shown that Sp1 and Sp3 were required for transactivation, and this was primarily dependent on proximal GC-rich motifs. Previous, studies have demonstrated that Sp4 was expressed in Panc-1 cells, and RNA interference assays suggested that Sp4 cooperatively interacted with Sp1 and Sp3 to activate VEGF promoter constructs in these cells. However, the relative contributions of Sp proteins to VEGF expression were variable among different pancreatic cancer cell lines. Small inhibitory RNAs for Sp3, but not Sp1 or Sp4, inhibited phosphorylation of retinoblastoma protein, blocked G₀/G₁→S-phase progression, and upregulated p27 protein/promoter activity of Panc-1 cells. Similar results were observed in other pancreatic cancer cells, suggesting that Sp3-dependent growth of pancreatic cancer cells is caused by inhibition of p27 expression (17). These data clearly demonstrate a critical role for Sp proteins for growth and angiogenesis of pancreatic cancer cells, and targeted degradation of these proteins would be highly advantageous for treatment of pancreatic cancer.

Nonsteroidal antiinflammatory drugs comprise large chemically heterogeneous groups of compounds which suppress inflammation by non-selectively inhibiting activity of cyclooxygenase-1 (COX-1) and cyclooxygenase-2 (COX-2) isoforms. Nonsteroidal antiinflammatory drugs are classified as belonging to one of the carboxylic acid groups, which includes diphenyl/diphenyl amine carboxylic acids, to one of the enolic acid groups or is classified as a coxib or as a gold salt. Generally, nonsteroidal antiinflammatory drugs alleviate pain and fever and, therefore, are used widely for the treatment of inflammatory disorders and conditions, such as rheumatoid arthritis, gout, bursitis, painful menstruation, and headache.

Studies have indicated that regularly taking aspirin or indomethacin, a carboxylic acid indole analog, provides a 40-50% reduction in relative risk of death by colon cancer (5). U.S. Pat. Nos. 6,207,700 and 6,399,647 describe a method of treating animals having cancer by administration of secondary amide derivatives of indomethacin. U.S. Pat. No. 5,914,322 discloses topical formulations of hyaluronic acid and an nonsteroidal antiinflammatory drug, such as diclofenac, indomethacin, naproxen, a trimethamine salt of ketorolac, ibuprofen, piroxicam, propionic acid derivatives, acetylsalicylic acid, and Flunixin are useful in treating primary and metastatic skin cancers and other skin disorders.

The role of NSAIDs in both prevention and treatment of colon cancer has been extensively investigated. Celecoxib, a coxib nonsteroidal antiinflammatory drug, has demonstrated antiangiogenic and antitumor activity against colon cancer (7). Hence, there is evidence from epidemiology studies that NSAIDs, such as aspirin and some COX-2 inhibitors, decreased the incidence and/or mortality of colon cancer. Patients with familial adenomatous polyposis (FAP) coli are highly susceptible for development of colon cancer and these individuals have been successfully treated with the COX-2 inhibitor sulindac (55-57). Laboratory animal and cell culture studies also confirm the efficacy of NSAIDs for inhibiting growth of colon cancer and tumors derived from other tissues (58-62).

It is also apparent that the anticancer activities of NSAIDs and COX-2 inhibitors can be both COX-2-dependent and -independent (48-52). Epidemiological studies on the association of NSAIDs with decreased risk/lower incidence of other cancers have been reported; however, the linkages are more variable and somewhat inconsistent (63-75). For example, several cohort studies report that breast cancer incidence is decreased with increasing aspirin/NSAID use in some cohorts but other studies indicate that aspirin and other NSAIDs may only provide minimal protection against breast cancer. A recent large cohort study concluded “that long duration regular NSAID use is associated with modestly reduced risk of prostate cancer” (73). Limited studies on pancreatic cancer suggest that decreased incidence of this disease was not correlated with aspirin/NSAID use (76-79).

NSAIDs/COX-2 inhibitors modulate several pathways in cancer cell lines that lead to inhibition of growth, apoptosis and antiangiogenesis, and COX-2 inhibitors are being investigated for colon cancer prevention and chemotherapy (63). Although prolonged use of NSAIDs may decrease incidence of some human cancers (chemoprevention), NSAIDs also exhibit antitumor activities in models for several cancers. For example, laboratory animal studies show that NSAIDs/COX-2 inhibitors such as aspirin, indomethacin, sulindac and celecoxib suppress carcinogen-induced or xenograft orthotopic models of lung, mast cell, fibrosarcoma, esophageal, bladder, pancreatic and mammary cancers (68-77). Although the mechanisms of these antitumorigenic effects induced by NSAIDs are not completely understood, there is strong evidence that NSAIDs inhibit cancer cell growth through modulation of cell cycle genes. Moreover, in combination with these antiproliferative properties, NSAIDs also induce apoptosis and exhibit antiangiogenic activities (48-50).

Wei and coworkers (8) first reported that celecoxib decreased cell/tumor growth, Sp1 and VEGF expression in pancreatic cancer cells and in tumors from nude mice bearing FG pancreatic cancer cells (orthotopic and xenograft models). A previous study using colon cancer cells as a model showed that celecoxib decreased Sp1 and Sp4 (but not Sp3) protein degradation and this was also accompanied by decreased expression of VEGF (2). Thus, the profile of NSAID-induced responses in cancer cells/tumors is highly desirable for an anticancer drug, for the development of NSAIDs (including COX-2 inhibitors) as a new class of mechanism-based drugs for treating pancreatic cancer.

Thus, the prior art is still deficient in cancer therapies employing nonsteroidal antiinflammatory drugs as antitumorigenic and antiangiogenic agents. More specifically, the prior art is deficient in chemotherapy regimens utilizing diphenyl/diphenyl amine carboxylic acids as therapeutic agents to degrade Sp proteins in cancer cells. The present invention fulfills this long-standing need ard desire in the art.

### SUMMARY OF THE INVENTION

The present invention is directed to a method of inducing degradation of one or more Sp family of transcription factors. Such a method comprises contacting a cancer cell with a non-steroidal anti-inflammatory drug, thereby inducing degradation of one or more Sp transcription factors.

The present invention is also directed to a method of treating a cancer in an individual. This method comprises administering a pharmacologically effective amount of diphenyl/diphenylamine carboxylic acid to the individual, thereby treating the cancer in the individual. The present invention is further directed to a method of treating pancreatic cancer in an individual. Such a method comprises administering a pharmacologically effective amount of tolfenamic acid, where the tolfenamic acid inhibits proliferation, angiogenesis and metastasis of the pancreatic cancer, thereby treating the pancreatic cancer in the individual.

The present invention is further directed to a method of reducing toxicity of a cancer therapy in an individual in need thereof. Such a method comprises administering to the individual a diphenyl/diphenylamine carboxylic acid and another chemotherapeutic drug, where the dosage of the chemotherapeutic drug administered is lower than the dosage required when said chemotherapeutic drug is administered singly, thereby reducing the toxicity of the cancer therapy in the individual.

The present invention is also directed to a method of treating cancer in an individual, consisting of administering a pharmacologically effective amount of diphenyl/diphenylamine carboxylic acid; and a siRNA specific for one or more Sp transcription factors, to the individual, thereby treating the cancer in the individual.

The present invention is further directed to a method of inhibiting an angiogenic response of a tumor in an individual consisting of administering a pharmacologically effective amount of diphenyl/diphenylamine carboxylic acid; and a siRNA specific for one or more Sp transcription factors, to the individual, thereby inhibiting the angiogenic response of the tumor in said individual. The present invention is also directed to a method of inhibiting tumor metastasis in an individual consisting of administering a pharmacologically effective amount of diphenyl/diphenylamine carboxylic acid; and a siRNA specific for one or more Sp transcription factors, to the individual, thereby inhibiting tumor metastasis in said individual.

### BRIEF DESCRIPTION OF THE DRAWINGS

FIG. 1A-1D show effects of nonsteroidal antiinflammatory drugs on Sp1, Sp3 and Sp4 protein expression in pancreatic cancer cells. FIG. 1A show results of screening of NSAIDs/COX-1/2 inhibitors for Sp protein degradation in Panc-1 cells. Cells were treated with 50 μM nonsteroidal antiinflammatory drugs/COX-1/2 inhibitors for 48 hr and whole cell lysates were analyzed by Western blot analysis. FIG. 1B shows quantitation of Sp proteins after treatment with nonsteroidal antiinflammatory drugs/COX-1/2 inhibitors. The results in FIG. 1A were determined in duplicate and the relative % Sp1, Sp3 and Sp4 levels in selected treated vs. control (DMSO; all values set at 100%) groups are presented as averages of 2 duplicate determinations. Protein band intensities were standardized based on β-tubulin protein as a loading control. HDAC protein is also shown and was unaltered by the treatments. Effects of selected NSAIDs on Sp protein in Panc-1 (FIG. 1C) and L3.6p1 cells (FIG. 1D) are also shown. Panc-1 cells were treated with DMSO, 50 μM ampiroxicam or tolfenamic acid for 24 or 48 hr, and Sp1, Sp3 and Sp4 protein levels were determined in whole cell lysates by Western blot analysis. Protein band intensities were normalized to β-tubulin and protein levels are presented as means±SE for 3 replicate determinations for each treatment group. Significantly (p<0.05) decreased protein levels are indicated by an asterisk.

FIGS. 2A-2G show decreased transactivation in pancreatic cancer cells transfected with pVEGF1 or pVEGF2 constructs and treated with nonsteroidal antiinflammatory drugs. Transfection of Panc-1 cells with pVEGF1 and pVEGF2 and treatment with 50 μM ampiroxicam and tolfenamic acid (FIGS. 2A, 2B) or diclofenac sodium (FIGS. 2C, 2D) with pVEGF1 or pVEGF2 constructs. Cells were transfected with pVEGF1 or pVEGF2 treated with DMSO, 50 μM ampiroxicam, tolfenamic acid, or diclofenac sodium, and luciferase activity determined. Transfection of L3.6p1 cells with pVEGF1 (FIG. 2E) or pVEGF2 constructs (FIG. 2F). Cells were transfected with pVEGF1 or pVEGF2 constructs, treated with DMSO, 50 μM ampiroxicam or tolfenamic acid, and luciferase activity determined. FIG. 2G shows concentration-dependent effects of nonsteroidal antiinflammatory drugs on transactivation. Panc-1 cells were transfected with pVEGF2, treated with 20, 40, 60 or 80 μM ampiroxicam or tolfenamic acid, and luciferase determined. Results presented herein are means±SE for three separate determinations per treatment group and a significant (p<0.05) decrease in luciferase activity is indicated by an asterisk.

FIGS. 3A-3B show effects of nonsteroidal antiinflammatory drugs on binding of nuclear extracts from Panc-1 cells to VEGF32P. Nuclear extracts from Panc-1 cells treated with DMSO, 50 μM ampiroxicam or tolfenamic acid for 48 hr were incubated with VEGF32P oligonucleotide alone (FIG. 3A) or in combination with various Sp antibodies (FIG. 3B) and analyzed by gel mobility shift.

FIGS. 4A-4D show nonsteroidal antiinflammatory drugs modulate VEGF expression in Panc-1 cells. FIG. 4A shows VEGF protein expression. Panc-1 cells were treated with DMSO, 50 μM ampiroxicam or tolfenamic acid for 48 hr and VEGF protein levels were determined by Western blot analysis. FIG. 4B shows VEGF mRNA levels. Panc-1 cells were treated with DMSO, 50 μM ampiroxicam and tolfenamic acid for 12 hr, and mRNA levels (relative to GAPDH) were determined by semiquantitative RT-PCR. Results in (FIG. 4A) and (FIG. 4B) are presented as means±SE for 3 separate determinations for each treatment group and significantly (p<0.05) decreased VEGF expression is indicated by an asterisk. FIG. 4C shows VEGF mRNA stability. Panc-1 cells were treated with actinomycin D (5 mg/ml) alone or in combination with 50 μM tolfenamic acid for up to 4.5 hr, and VEGF mRNA levels were determined at time 0 and 1.5, 2.5 and 4.5 hr after treatment by semiquantitative RT-PCR. Results are the average of duplicate experiments, and VEGF mRNA levels are normalized to GADPH mRNA. FIG. 4D show immunostaining of VEGF. Panc-1 cells were treated with DMSO, 50 μM ampiroxicam and tolfenamic acid for 48 hr and immunostaining for VEGF was determined.

FIGS. 5A-5B show effects of the proteasome inhibitor lactacystin on tolfenamic acid-induced degradation of Sp proteins and activation of pVEGF2. FIG. 5A shows that lactacystin inhibits degradation of Sp proteins. Panc-1 cells were treated with DMSO, 50 μM ampiroxicam or tolfenamic acid alone or in combination with 2 μM lactacystin for 48 hr, and whole cell lysates were analyzed for Sp proteins by Western blot analysis. FIG. 5B shows that lactacystin inhibits decreased transactivation in cells transfected with pVEGF2 construct. Panc-1 cells were transfected with pVEGF2 construct, treated with DMSO, 50 μM ampiroxicam and tolfenamic acid alone or in combination with 2 μM lactacystin, and luciferase activity determined. Results are expressed as means±SE for three separate determinations for each treatment group and significantly (p<0.05) decreased activity by tolfenamic acid (*) or inhibition of this response by lactacystin (**) are indicated.

FIGS. 6A-6E show comparative effects of nonsteroidal antiinflammatory drugs on Panc-1 cell proliferation and Sp protein expression. Effects of tolfenamic acid (FIG. 6A), ampiroxicam (FIG. 6B), naproxen (FIG. 6C), and diclofenac sodium (FIG. 6D) on Panc-1 cell proliferation. Panc-1 cells were treated with different concentrations of nonsteroidal antiinflammatory drugs for 6 days, and the number of cells were determined on days 2, 4, and 6 by cell counting techniques. Results are expressed as means of at least three replicates for each determinations, and the SE values were <15% for all time points. Similar results were obtained using the WST assay for ampiroxicam and tolfenamic acid (data not shown). FIG. 6E shows NSAID-induced apoptosis and degradation of Sp proteins. Panc-1 cells were treated with DMSO, 150 μM ampiroxicam, 150 μM naproxen, and 50 μM tolfenamic acid for 48 hr, and levels of Sp proteins and the cleaved PARP protein were determined in whole cell lysates by Western blot analysis. The concentrations of nonsteroidal antiinflammatory drugs used in the experiment corresponded to doses that exhibited comparable growth inhibitory activities.

FIGS. 7A-7D compares effects of tolfenamic acid and ampiroxicam on pancreatic cancer cell proliferation. L3.6p1 (FIGS. 7A, 7B), and Panc-28 (FIGS. 7C, 7D) cells were treated with DMSO, 25, 50 or 100 μM ampiroxicam or tolfenamic acid for 6 days, and every 2 days, cells were counted. Results are expressed as means±SE for 3 separate determinations for each treatment group and tolfenamic acid significantly (p<0.05) inhibited cell growth at concentrations of 25-100 μM.

FIGS. 8A-8E show inhibition of pancreatic tumor growth and angiogenesis by tolfenamic acid and gemcitabine. Decreased tumor volume (FIG. 8A) and weight (FIG. 8B). Median tumor volumes (FIG. 8A) and weights (FIG. 8B) in athymic nude mice treated orthotopically with L3.6p1 cells, and corn oil (control), tolfenamic acid (25 and 50 mg/kg), and gemcitabine (50 mg/kg) were determined. Results are expressed as means±SD and significantly (p<0.05) decreased tumor volumes and weights compared to the corn oil control are indicated by an asterisk. FIG. 8C shows Sp and VEGF protein expression. Tumors from the various treatment groups were analyzed for Sp1, Sp3, Sp4 and VEGF protein expression by Western blot analysis. Results are expressed as means±SE for at least three separate determinations for each treatment group and expressed relative to the solvent (corn oil) control and normalized to β-tubulin within each group. Immunostaining for VEGF (FIG. 8D) and CD31 (FIG. 8E). Pancreatic tumor sections from animals treated with solvent (control), gemcitabine (5C mg/kg) and tolfenamic acid (25 and 50 mg/kg) were immunostained with VEGF and CD31 antibodies.

FIGS. 9A-9B show Sp protein domains. FIG. 9A shows structural features of Sp protein domains that is used to prepare Gal4/Spx-y constructs. FIG. 9B shows lysine residue distribution in Sp protein. Numbers represent aminoacid sites according to Refseq accession number for Sp proteins.

FIG. 10 shows Gal4/Spx-y constructs.

FIG. 11A-11C shows VEGF (FIG. 11A), VEGFR1 (FIG. 11B) and VEGFR2 (FIG. 11C) constructs.

FIG. 12 shows GC-rich p27 promoter construct.

FIGS. 13A-13D show VEGFR1 expression in pancreatic cancer cells is Sp protein dependent. Transfection with pVEGFR1-A (FIG. 13A), pVEGFR1-B (FIG. 13B), and pVEGFR1-C (FIG. 13C) and small inhibitory RNAs for Sp1 (iSp1), Sp3 (iSp3) or Sp4 (iSp4) (FIG. 13D). Panc-1 cells were transfected with the pVEGFR1 constructs and iSp1, iSp3 or iSp4, and luciferase activity was determined. As a control for the transfection experiment, cells were transfected with a non-specific small inhibitory RNA (iNS) and luciferase activity for transfection with iNS was set at 100%. All experiments were replicated at least three times and results are expressed as means±SD. Significantly (p<0.05) decreased luciferase activity is indicated (*). Effects of iSp1, iSp3 and iSp4 on Sp and VEGFR1 proteins (FIG. 13D). Panc-1 cells were transfected with iNS, iSp1, iSp3 or iSp4, and whole cell lysates were analyzed by Western blot analysis. The experiment was replicated (3X) and relative expression of individual Sp proteins and VEGFR1 were compared to levels in cells treated with iNS (set at 100%). Significantly (p<0.05) decreased protein expression is indicated by an asterisk.

FIGS. 14A-14D show Tolfenamic acid decreases Sp and VEGFR1 proteins in pancreatic cancer cells. Effects of tolfenamic acid in Panc-1 (FIG. 14A and FIG. 14B) and L3.6p1 (FIG. 14C and D) cells. Cells were treated with DMSO, 50 μM tolfenamic acid, or 50 μM ampiroxicam for 48 h, and whole cell lysates were analyzed by Western blot analysis. The experiment was replicated (3X) and the Sp and VEGFR1 protein levels were set at 100% and significantly (p<0.05) decreased expression of Sp1, Sp3, Sp4 and VEGFR2 is indicated by an asterisk.

FIG. 15 shows immunohistochemical analysis of VEGFR1 in pancreatic cancer cells treated with tolfenamic acid. Panc-1 and L3.6p1 cells were treated with DMSO, 50 μM ampiroxicam, or 50 μM tolfenamic acid for 48 h, and cells were immunostained by VEGFR1 antibodies.

FIGS. 16A-16D show Interactions of Sp proteins with VEGFR1 promoter sequences. Panc-1 cells were treated with DMSO, 50 μM ampiroxicam, or 50 μM tolfenamic acid for 48 h, and nuclear extracts were incubated with VEGFR1³²P (FIG. 16A) or GC/Sp³²P (FIG. 16B) oligonucleotides in the presence or absence of Sp1, Sp3 or Sp4 antibodies and analyzed in a gel mobility shift assay. The specific Sp protein bands and antibody supershifted complexes are indicated with arrows. Effects of tolfenamic acid on VEGFR1 and Egr-1 protein expression in Panc-1 (FIG. 16C) and L3.6p1 (FIG. 16D) cells. Cells were treated with DMSO, 50 μM ampiroxicam, or 50 μM tolfenamic acid for 48 h, and whole cell lysates were examined by Western blot analysis.

FIGS. 17A-17D show effects of tolfenamic acid of VEGFR1 promoter and mRNA expression. Transfection with pVEGFR1-A (FIG. 17A), pVEGFR1-B (FIG. 17B), and pVEGFR1-C (FIG. 17C). Panc-1 cells were transfected with pVEGFR1 constructs, treated with DMSO, 50 μM ampiroxicam, or 50 μM tolfenamic acid, and luciferase activity was determined described in the Materials and Methods. Results are expressed as means±SD for replicate (3) experiments for each treatment group and significantly (p<0.05) decreased activity is indicated by an asterisk. (FIG. 17D) Decreased VEGFR1 mRNA in Panc-1 and L3.6p1 cells. Panc-1 or L3.6p1 cells were treated with DMSO, 50 μM ampiroxicam, or 50 μM tolfenamic acid for 24 h, and relative mRNA expression was determined by semiquantitative reverse transcription PCR. Results are illustrated for a single experiment and similar data was obtained in a replicate experiment.

FIGS. 18A-18D show tolfenamic acid inhibits activation of VEGFR1 in pancreatic cancer cells. Inhibition of Erk1/2 phosphorylation in Panc-1 (FIG. 18A) and L3.6p1 (FIG. 18B) cells. Cells were pretreated with DMSO, 50 μM ampiroxicam, or 50 μM tolfenamic acid for 36 hr; VEGF-B (50 ng/ml) was added for 5 or 10 min, and whole cell lysates were obtained and analyzed by Western blot analysis. Cell migration assay (FIG. 18C). Panc-1 cells were treated with DMSO, 50 μM ampiroxicam, or 50 μM tolfenamic acid for 24 h. VEGF-B (50 ng/ml) was added and the inhibition of cell migration (relative to DMSO-treated cells set at 100%) was determined 16 h after addition of VEGF-B. The experiments were replicated (3×) and results are expressed as means±SD and significantly (p<0.05) decreased cell migration is indicated (*). Immunostaining of pancreatic tumors (FIG. 18D). Pancreatic tumors from athymic nude mice treated with solvent (control), gemcitabine (50 mg/kg), or tolfenamic acid (25 and 50 mg/kg) (27) were stained with VEGFR1 antibodies as described below and in a previous report (27).

### DETAILED DESCRIPTION OF THE INVENTION

The present invention demonstrates that chemical-induced degradation of Sp proteins by the diphenyl/diphenylamine carboxylic acid subclass of nonsteroidal antiinflammatory drugs inhibited growth and angiogenesis in cancer cells. The present invention demonstrated that tolfenamic acid and structurally related non-steroidal anti-inflammatory drugs specifically activate proteosome-dependent degradation of Sp family transcription factors (Sp1, Sp3 and Sp4) in cancer cells and thereby inhibited cell proliferation through induction of p27 and also decreased Sp-dependent expression of the angiogenic factor VEGF. These are the only compounds that that induced degradation of Sp1, Sp3 and Sp4. By using in vitro and in vivo experiments, the present invention demonstrated that these compounds inhibited pancreatic cell and tumor growth, pancreatic tumor angiogenesis and liver metastasis.

VEGF and related angiogenic growth factors and their receptors play a critical role in tumorigenesis and contribute significantly to cancer cell progression and metastasis. Not surprisingly, VEGF/VEGFR signaling pathways have been extensively targeted for cancer chemotherapy. Antiangiogenic compounds initially discovered have multiple mechanisms of action; however, several alternative approaches have also been reported and these include antibodies that block VEGF and/or VEGFR and tyrosine kinase inhibitors that block VEGFR kinase signaling. Other approaches include development of arginine-rich peptides that block VEGF action or by blocking downstream factors such as Src family kinases that mediate some of the VEGFR1-dependent responses in colon cancer cells. A construct expressing domains of both VEGFR1 and VEGFR2 that tightly binds VEGF through the extracellular domain of VEGFR-1 (VEGF-Trap) has also been used to inhibit tumor growth and metastasis in animal models.

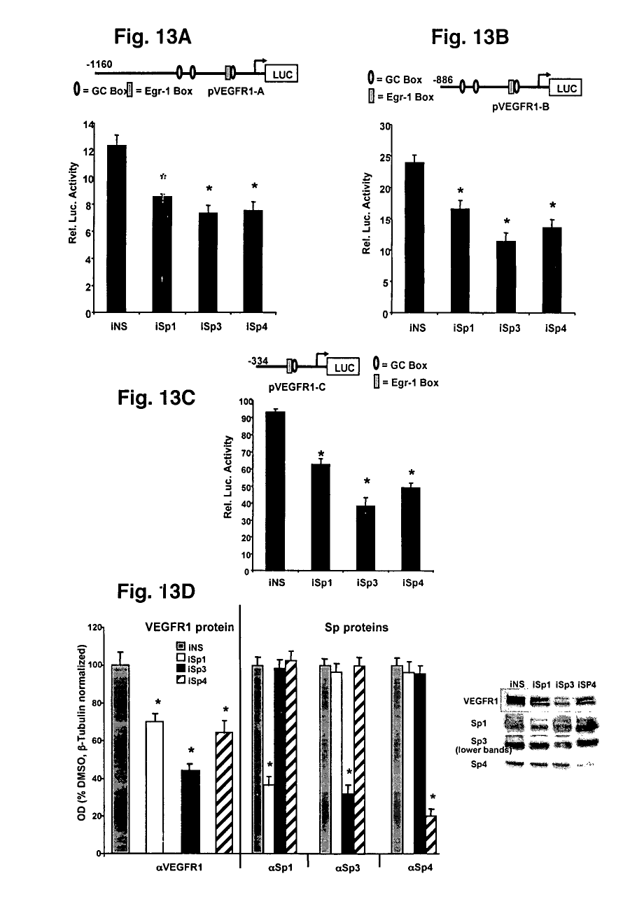

Previous studies have shown that expression of both VEGF and VEGFR2 in pancreatic and other cancer cell lines was regulated by Sp1, Sp3 and Sp4, and RNA interference with small inhibitory RNAs targeting these proteins decreased VEGF and VEGFR2 expression. The instant invention has demonstrated that, in pancreatic cancer cells and tumors, tolfenamic acid induced proteasome-dependent degradation of Sp1 and Sp3 and Sp4, and not surprisingly, tolfenamic acid inhibited angiogenesis and decreased liver metastasis in an orthotopic model of pancreatic cancer using the highly metastatic L3.6p1 cell. Since VEGFR1 also plays a pivotal role in pancreatic tumor migration and invasion, the present invention investigated the molecular mechanism of VEGFR1 regulation in pancreatic cancer cells. Takeda and coworkers identified VEGFR1 as a gene regulated by the basic helix-loop-helix endothelial PAS domain protein 1 (EPAS1) which forms a heterodimer with hypoxia-inducible factor 1B to activate VEGFR1 and other proangiogenic gene. However, the VEGFR1 gene has three consensus GC-rich motifs that bind Sp proteins, and results from RNA interference studies show that knockdown of Sp1, Sp3 or Sp4 also decreased VEGFR1 protein expression in pancreatic cancer cells (FIG. 13). Moreover, similar results were observed using a series of deletion constructs containing VEGFR1 promoter inserts (FIG. 13).

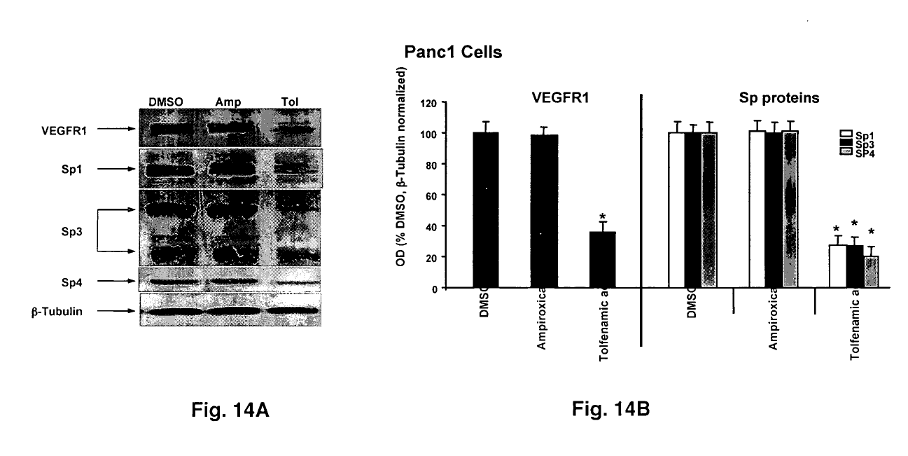

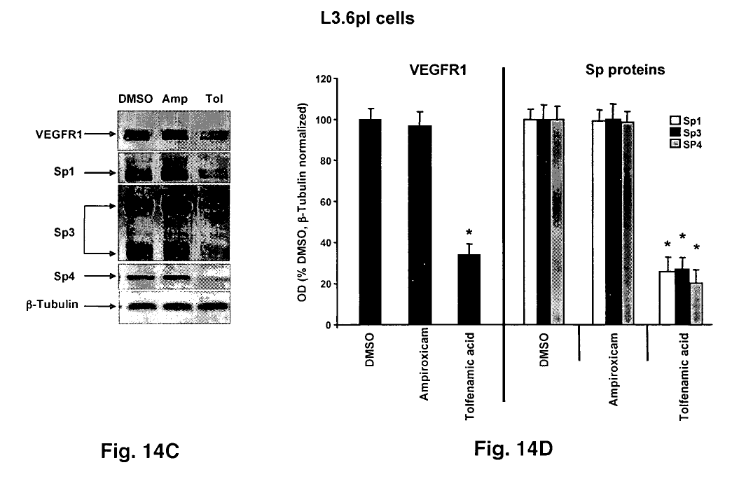

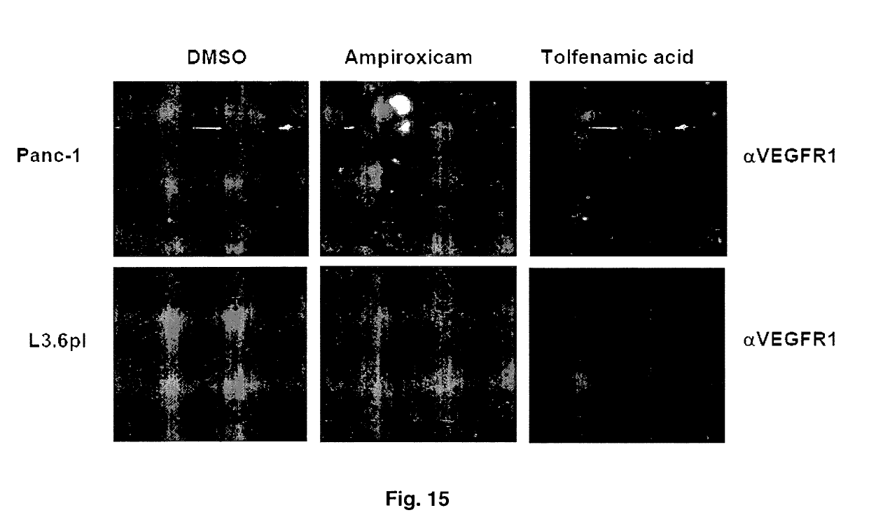

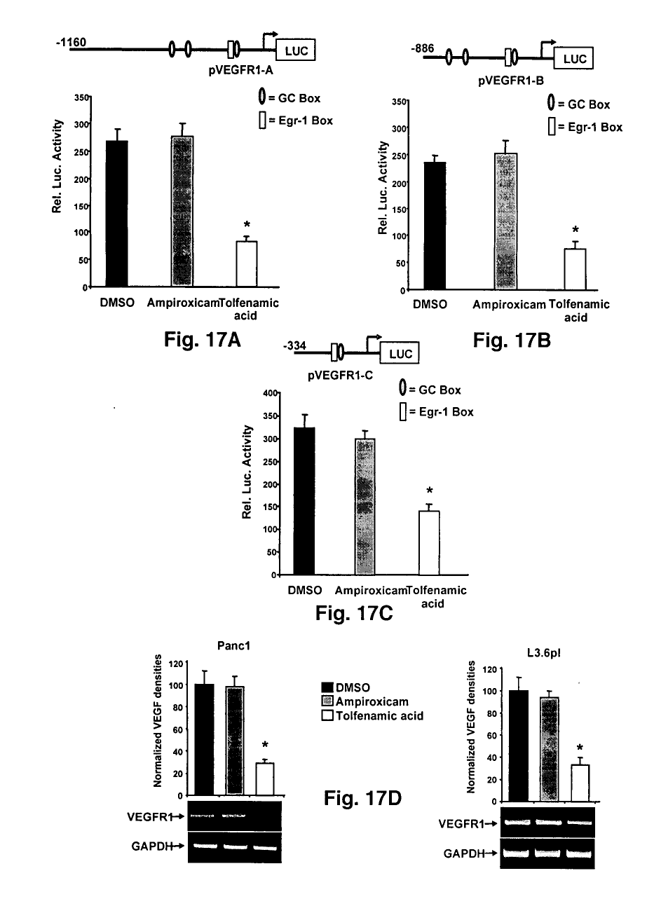

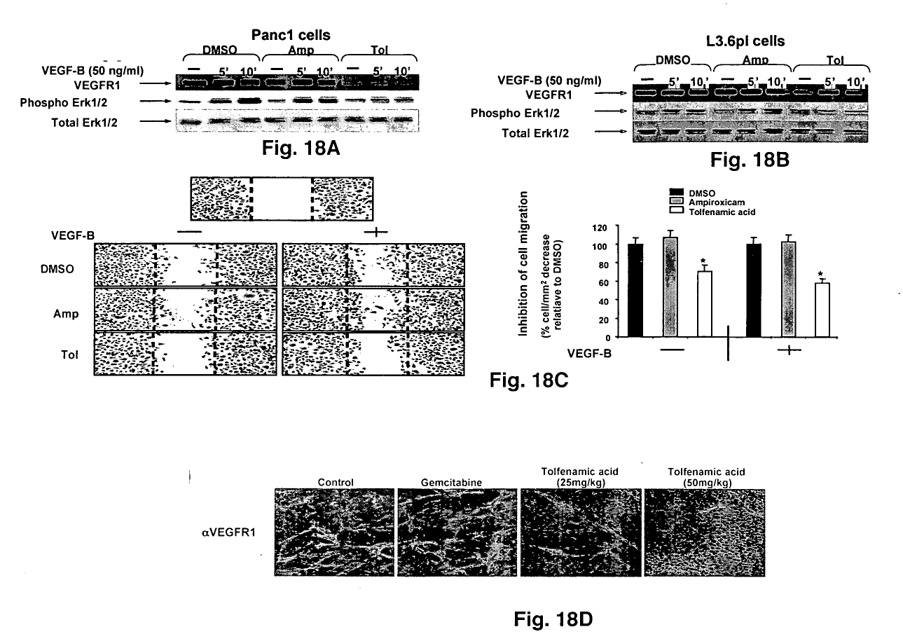

These results suggest that like VEGF and VEGFR, VEGFR1 expression in pancreatic cancer cells is Sp-dependent and therefore compounds such as tolfenamic acid, which decrease Sp1, Sp3 and Sp4 (FIG. 15) should also decrease VEGFR1. This was confirmed in a series of experiments showing that tolfenamic acid but not the NSAID ampiroxicam [a negative control, decreases VEGFR1 protein (FIGS. 14A, 14C, 15 and 16), and mRNA (FIG. 17D) as well as luciferase activity in cells transfected with VEGFR1 constructs (FIGS. 17A-17B). Moreover, decreased VEGFR1 expression (FIG. 18D) was observed in pancreatic tumors from mice treated with tolfenamic acid and this was accompanied by decreased levels of VEGF and Sp protein in these tumors. Since VEGFR1 mediates VEGFB-induced migration and invasion of pancreatic and colon cancer cells, we also investigated the role of Sp proteins in mediating Panc-1 cell migration in a “scratch” test in monolayer cultures grown on collagen IV coated plates (FIG. 18C). Previous reports show that VEGF-A and VEGF-B induce migration of colon and pancreatic cancer cells using a Boyden chamber assay, and results in FIG. 18C confirm that VEGF-B also induced migration of Panc1 cells. Inhibition of VEGF-B-induced migration of colon and pancreatic cancer cells was observed in cells treated with the VEGFR1 antibody (18F1), and similar inhibitory effects were observed in this study in Panc1 cells cotreated with VEGF-B plus tolfenamic acid (FIG. 18C). In contrast, tolfenamic acid also inhibited Panc1 cell migration in solvent-treated (DMSO) cells, whereas the VEGFR1 antibody did not affect migration of untreated/solvent control cells. The differences between tolfenamic acid and VEGFR1 antibodies must be due to the overall decrease in VEGFR1 levels (FIG. 14) in cells treated with tolfenamic acid, whereas the antibodies do not affect VEGFR1 expression. VEGF-B-induced phosphorylation of MAPK was also examined in Panc1 and L3.6p1 cells and, like the VEGFR1 antibody, tolfenamic acid decreased MAPK phosphorylation in Panc1 and L3.6p1 cells (FIGS. 18A and 18B). This was consistent with the parallel downregulation of VEGFR1 protein in these cells. Thus, the inhibition of VEGF-B-induced migration of pancreatic cancer cells by tolfenamic acid is also accompanied by inhibition of VEGFR1-dependent downstream signaling. Ampiroxicam, an NSAID that does not induce Sp protein degradation in Panc1 cells, was also used as a control in the cell migration and MAPK phosphorylation studies, and this compound was inactive in all assays.

The results discussed herein show that cancer cell growth and angiogenesis can be targeted via the Sp proteins. Furthermore, although the inhibitory effects of these compounds were demonstrated in pancreatic cancer cells and pancreatic tumors, similar effects were also observed in breast, prostate, colon, bladder and ovarian cancer cells. Overall, tolfenamic acid and structurally-related nonsteroidal antiinflammatory drugs containing biphenyl and diphenylamine carboxylic acid structures alone or in combination with other cancer chemotherapeutic drugs provides the advantage of using relatively non-toxic well-tested drugs for inhibiting tumor growth and angiogenesis by degrading all three Sp proteins (Sp1, Sp3 and Sp4) that are critical for tumor/cancer cell growth and metastasis.

In one embodiment of the present invention, there is provided a method of inducing degradation of one or more Sp transcription factors, comprising: contacting a cancer cell with a non-steroidal anti-inflammatory drug thereby inducing degradation of one or more Sp transcription factors. The degradation of Sp transcription factors may induce the expression of p27 such that proliferation of the cancer cell is inhibited. The degradation may also decrease the expression of VEGF such that angiogenesis, tumor metastasis or a combination thereof in a tumor comprising the cancer cell is inhibited. Preferably, the nonsteroidal antiinflammatory drug is a diphenyl/diphenylamine carboxylic acid. Representative examples of such diphenyl/diphenylamine carboxylic acid includes but is not limited to tolfenamic acid, diclofenac sodium and diflunisal. Moreover, the Sp transcription factor is a Sp1 protein, Sp3 protein, Sp4 protein or a combination thereof. Additionally, example of the cancer cell includes but is not limited to a pancreatic cancer cell, a breast cancer cell, a prostate cancer cell, a colon cancer cell, a bladder cancer cell or an ovarian cancer cell.

In another embodiment of the present invention, there is provided a method of treating cancer in an individual, comprising: administering a pharmacologically effective amount of diphenyl/diphenylamine carboxylic acid to the individual, thereby treating the cancer in the individual. This method may further comprise administering another chemotherapeutic drug. Such a chemotherapeutic drug may be administered concurrently or sequentially with the diphenyl/diphenylamine carboxylic acid. Furthermore, such a diphenyl/diphenylamine carboxylic acid may inhibit tumor growth, angiogenesis and metastasis in the individual. Examples of such diphenyl/diphenylamine carboxylic acid includes but is not limited to tolfenamic acid, diclofenac sodium, and diflunisal. Additionally, an individual benefiting from such a method includes but is not limited to one who is diganosed with pancreatic cancer, breast cancer, prostate cancer, colon cancer, bladder cancer or ovarian cancer.

In yet another embodiment of the present invention, there is provided a method of treating pancreatic cancer in an individual, comprising: administering a pharmacologically effective amount of tolfenamic acid to the individual, where the tolfenamic acid inhibits proliferation, angiogenesis and metastasis of the pancreatic cancer, thereby treating the pancreatic cancer in the individual. This method may further comprise administering another chemotherapeutic drug. Such a chemotherapeutic drug may be administered concurrently or sequentially with the tolfenamic acid.

In another embodiment of the present invention, there is provided a method of reducing toxicity of a cancer therapy in an individual in need thereof, comprising: administering to the individual a diphenyl/diphenylamine carboxylic acid and another chemotherapeutic drug, where the dosage of the chemotherapeutic drug administered is lower than the dosage required when the chemotherapeutic drug is administered singly, thereby reducing the toxicity of the cancer therapy in the individual. The chemotherapeutic drug may be administered concurrently or sequentially with the diphenyl/diphenylamine carboxylic acid. Examples of such chemotherapeutic drug include but is not limited to and that of the diphenyl/diphenylamine carboxylic acid include and is not limited to tolfenamic acid, diclofenac sodium, and diflunisal. Furthermore, examples of the cancer includes and is not limited to pancreatic cancer, breast cancer, prostate cancer, colon cancer, bladder cancer or ovarian cancer.

In yet another embodiment of the present invention there is a method of treating cancer in an individual, consisting of administering a pharmacologically effective amount of diphenyl/diphenylamine carboxylic acid; and a siRNA specific for one or more Sp transcription factors, to the individual, thereby treating the cancer in the individual. In general, the treatment inhibits tumor growth, angiogenesis and/or metastasis in the individual. Specifically, the diphenyl/diphenylamine carboxylic acid is tolfenamic acid, diclofenac sodium, or diflunisal. The individual is diagnosed with pancreatic cancer, breast cancer, prostate cancer, colon cancer, bladder cancer or ovarian cancer. Specifically, the Sp transcription factor is a Sp1 protein, a Sp3 protein, a Sp4 protein or a combination thereof In still yet another embodiment of the present invention there is a method of inhibiting the angiogenic response of a tumor in an individual consisting of administering a pharmacologically effective amount of diphenyl/diphenylamine carboxylic acid; and a siRNA specific for one or more Sp transcription factors, to the individual, thereby inhibiting the angiogenic response of the tumor in said individual. In general, this method results in decrease in expression of VEGFR1, such that angiogenic response of the tumor is inhibited. Moreover, this method inhibits tumor growth and metastasis in the individual. Specifically, the diphenyl/diphenylamine carboxylic acid is tolfenamic acid, diclofenac sodium, or diflunisal. In general, the individual has a pancreatic cancer, breast, prostate, colon, bladder or ovarian tumor.

In still yet another embodiment of the present invention there is a method of inhibiting tumor metastasis in an individual consisting of administering a pharmacologically effective amount of diphenyl/diphenylamine carboxylic acid; and a siRNA specific for one or more Sp transcription factors, to the individual, thereby inhibiting tumor metastasis in said individual. In general, the method decreases the expression of VEGFR1, such that tumor metastasis is inhibited. Specifically, the diphenyl/diphenylamine carboxylic acid is tolfenamic acid, diclofenac sodium, or diflunisal. In general, the individual has a pancreatic cancer, breast, prostate, colon, bladder or ovarian tumor.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “contacting” refers to any suitable method of bringing an nonsteroidal antiinflammatory drug into contact with a cancer cell. In vitro or ex vivo this is achieved by exposing the cancer cell to the nonsteroidal antiinflammatory drug in a suitable medium. For in vivo applications, any known method of administration is suitable as described herein.

As used herein, the term “treating” or the phrase “treating a cancer” includes, but is not limited to, halting the growth of the cancer, killing the cancer, or reducing the size of the cancer. Halting the growth refers to halting any increase in the size or the number of or size of the cancer cells or to halting the division of the neoplasm or the cancer cells. Reducing the size refers to reducing the size of the cancer or the number of or size of the cancer cells.

Another chemotherapeutic drug may be administered concurrently or sequentially with the nonsteroidal antiinflammatory drug used herein. The effect of co-administration with the nonsteroidal antiinflammatory drug is to lower the dosage of the chemotherapeutic drug normally required that is known to have at least a minimal pharmacological or therapeutic effect against a cancer or cancer cell, for example, the dosage required to eliminate a cancer cell. Concomitantly, toxicity of the chemotherapeutic drug to normal cells, tissues and organs is reduced without reducing, ameliorating, eliminating or otherwise interfering with any cytotoxic, cytostatic, apoptotic or other killing or inhibitory therapeutic effect of the drug on the cancer cells.

Nonsteroidal antiinflammatory drugs used herein and other chemotherapeutic drugs can be administered independently, either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant. Dosage formulations of the nonsteroidal antiinflammatory drugs may comprise conventional non-toxic, physiologically or pharmaceutically acceptable carriers or vehicles suitable for the method of administration.

The nonsteroidal antiinflammatory drugs and chemotherapeutic drugs or pharmaceutical compositions thereof may be administered independently one or more times to achieve, maintain or improve upon a therapeutic effect. It is well within the skill of an artisan to determine dosage or whether a suitable dosage of either or both of the nonsteroidal antiinflammatory drugs and chemotherapeutic drug comprises a single administered dose or multiple administered doses. An appropriate dosage depends on the subject's health, the progression or remission of the cancer, the route of administration and the formulation used.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE1

### Cell Lines Chemicals, Biochemical, Constructs and Oligonucleotides

Panc-1 cells were obtained from the American Type Culture Collection (ATCC, Manassas, Va.). L3.6p1 cell line was developed in at the M. D. Anderson Cancer Center (Houston, Tex.) and provided by Dr. I. J. Fidler. VEGFR1 promoter luciferase constructs were provided by Dr. Koji Maemura (Department of Cardiovascular Medicine, University of Tokyo, Japan). DME/F12 with and without phenol red, 100× antibiotic/antimycotic solution and lactacystin were purchased from Sigma Chemical Co. (St. Louis, Mo.). Collagen IV-coated plates were purchased from Becton Dickinson Labware (Bedford, Mass.). Diff Quik staining kit was obtained from Dade Behring (Newark, Del.). Fetal bovine serum was purchased from Intergen (Purchase, N.Y.). [γ-³²P]ATP (300 Ci/mmol) was obtained from Perkin Elmer Life Sciences. Poly (dI-dC) and T4 polynucleotide kinase were purchased from Roche Molecular Biochemicals (Indianapolis, Ind.). Antibodies for Sp1, Sp3, Sp4, HDAC, β-tubulin and VEGFR1 proteins were obtained from Santa Cruz Biotechnology (Santa Cruz, Calif.). ERK1/2 and pERK12/ were obtained from Zymed Laboratories Inc. (San Francisco, Calif.). Lysis buffer and luciferase reagent were obtained from Promega Corp. (Madison, Wis.).

### EXAMPLE 2

### Transfection of Pancreatic Cancer Cells and Preparation of Nuclear Extracts

Cells are cultured in 6-well plates in 2 ml of DME/F12 medium supplemented with 5% fetal bovine serum. After 16-20 hr when cells are 50-60% confluent, reporter gene constructs are transfected using lipofectamine Reagent (Invitrogen, Carlsbad, Calif.). The effects of the selective NSAIDs on transactivation are investigated in Panc1 and L3.6p1 cells cotransfected with different VEGF constructs (500 ng). Cells are treated with DMSO (control) or with the indicated concentration of NSAIDs for 24 and/or 48 hr, then luciferase activity of lysates (relative to β-galactosidase activity) are determined. For proteasome inhibitor experiments, cells will be cotreated with 2 μM lacatacystin, and for EMSA assays, nuclear extracts from Panc1 and L3.6p1 cells are isolated as previously described, and aliquots will be stored at −80° C. until used (17, 19).

### EXAMPLE 3

### Western Immunoblot

Cells are washed once with PBS and collected by scraping in 200 μL of lysis buffer [50 mM HEPES, 0.5 M sodium chloride, 1.5 mM magnesium chloride, 1 mM EGTA, 10% (v/v) glycerol, 1% Triton X-100, 5 μL/ml of Protease Inhibitor Cocktail (Sigma)]. The lysates from the cells are incubated on ice for 1 hr with intermittent vortexing followed by centrifugation at 40,000 g for 10 min at 4° C. Equal amounts of protein (60 μg) from each treatment group are diluted with loading buffer, boiled and loaded onto 10 and 12.5% SDS-polyacrylamide gel. For VEGF immunoblots, 100 μg of protein are used. Samples are electrophoresed and proteins detected by incubation with polyclonal primary antibodies Sp1 (PEP2), Sp3 (D-20), Sp4 (V-20), HDAC (H-5 1), VEGF (a-20) and β-tubulin (H-235) followed by blotting with appropriate horseradish peroxidase-conjugated secondary antibody as previously described (17). After autoradiography, band intensities are determined by a scanning laser densitometer (Sharp Electronics Corporation, Mahwah, N.J.) using Zero-D Scanalytics software (Scanalytics Corporation, Billerica, Mass.).

### EXAMPLE 4

### Electrophoretic Mobility Shift Assay (EMSA)

VEGF and VEGFR1 oligonucleotides are synthesized and annealed, and 5-pmol aliquots are 5′-end-labeled using T4 kinase and [γ-³²P]ATP. The EMSA reaction mixture (30 μL) contains ˜100 mM KC1, 3 μg of crude nuclear protein, 1 μg poly (dI-dC), with or without unlabeled competitor oligonucleotide, and 10 fmol of radiolabeled probe. After incubation for 20 min on ice, antibodies against Sp1, Sp3 and Sp4 proteins are added and incubated another 20 min on ice; antibodies against Sp1, Sp3 and Sp4 proteins are added and incubated another 20 min on ice. Protein-DNA complexes are resolved by 5% polyacrylamide gel electrophoresis as previously described (17, 19, 23). Specific DNA-protein and antibody-supershifted complexes are observed as retarded bands in the gel.

### EXAMPLE 5

### Cell Proliferation Assay

Panc1, Panc28 and L3.6p1 cells are seeded in DMEM/F12 media with 5% FBS and treated the next day with either vehicle (DMSO) or with the indicated compounds' concentrations. Cells are counted at the indicated times using a Coulter Z1 cell counter. Each experiment is carried out in triplicate and results expressed as means±SD for each determination.

### EXAMPLE 6

### VEGF-B Activation of MAPK and Cell Migration

Panc-1 cells were pretreated with DMSO, 50 μM ampiroxicam, or 50 μM tolfenamic acid for 48 h, then treated with VEGF-B (50 ng/ml) for 5 and 10 min. MAPK phosphorylation in the various treatment groups was then determined by Western immunoblot analysis as described above. For migration assays, Panc-1 cells were seeded in triplicates in six-well collagen IV coated plates and then treated with the selected NSAIDs for 24 h before the scratch was made. A scratch through the central axis of the plate was gently made using a sterile pipette tip. Cells were 70% confluent when the scratch was made. Cells were then washed and treated with the DMSO control, selected NSAIDs alone, or NSAIDs and VEGF-B. Migration of the cells into the scratch was observed at nine preselected points (three points per well) at 0, 8, and 16 h. Results of this study were obtained at a 16 h time point and one plate was stained using Diff Quik (Dade Behring, Newark, Del.).

### EXAMPLE 7

### Immunocytochemistry

Panc1 cells are seeded in Lab-Tek Chamber slides (Nalge Nunc International, Naperville, Ill.) at 100,000 cells/well in DME/F12 medium supplemented with 5% fetal bovine serum. Cells are treated with the selected NSAIDs and after 48 hr, the media chamber is detached and the remaining glass slides washed in Dulbecco's PBS. The immunostaining for VEGF is determined essentially as previously described (19). Briefly, the glass slides are fixed with cold (−20° C.) methanol for 10 min and then washed in 0.3% PBS/Tween for 5 min (2×) before blocking with 5% goat serum in antibody dilution buffer (stock solution: 100 ml of PBS/Tween, 1 g of bovine serum albumin, 45 ml of glycerol, pH 8.0) for 1 hr at 20° C. After removal of the blocking solution, VEGF rabbit polyclonal antibody (A-20) is added in antibody dilution buffer (1:200) and incubated for 12 hr at 4° C. Slides are washed for 10 min with 0.3% Tween in 0.02 M PBS (3×) and incubated with fluorescein isothiocyanate-conjugated (FITC) goat anti-rabbit antibodies (1:1000 dilution) for 2 hr at 20° C. Slides are then washed for 10 min in 0.3% PBS-Tween (4×). Slides are mounted in ProLonged antifading medium with DAPI for nuclear counterstaining (Molecular Probes, Inc., Eugene, Oreg.) and cover slips sealed using Nailslicks nail polish (Noxell Corp., Hunt Valley, Md.).

### EXAMPLE 8

### Semiquantitative Reverse Transcription-PCR Analysis

Panc1 cells are treated with DMSO (control) or with the indicated concentration of NSAIDs for 48 hr before total RNA collection. Total RNA is obtained with RNAzol B (Tel-Test, Friendswood, Tex.) according to the manufacturer's protocol. RNA concentration is measured by UV 260:280 nm absorption ratio, and 200 ng/μL RNA is used in each reaction for reverse transcription-PCR. RNA is reverse transcribed at 42° C. for 25 min using oligo d(T) primer (Promega) and subsequently PCR amplified of reverse transcription product using 2 mmol/L MgCl₂, 1 μmol/L of each gene-specific primer, 1 μmol/L dNTPs, and 2.5 units AmpliTaq DNA polymerase (Promega). The gene products are amplified using 22 to 25 cycles (95° C., 30 s; 56° C., 30 s; 72° C., 30 s). The sequences of the oligonucleotide primers in are: VEFG forward 5′-CCA TGA ACT TTC TGC TGT CTT-3′, VEGF reverse 5′-ATC GCA TCA GGG GCA CAC AG-3′, GAPDH forward 5′-AATCCCATCACCATCTTCCA-3′, and GAPDH reverse 5′-GTCATCATATTTGGCAGGTT-3′, VEGFR1 forward: 5′-TGG GAC AGT AGA AAG GGC TT-3′, VEGFR1 reverse: 5′-GGT CCA CTC CTT ACA CGA CAA -3′, GAPDH forward: 5′-AAT CCC ATC ACC ATC TTC CA-3′, GAPDH reverse: 5′-GTC ATC ATA TTT GGC AGG TT-3′.

Following amplication in a PCR express thermal cycler (Hybaid US, Franklin, Mass.), 20 μL of each sample is loaded on a 2% agarose gel containing ethidium bromide. Electrophoresis is performed at 80 V in 1×TAE buffer for 1 hr and the gel photographed by UV transillumination using Polaroid film (Waltham, Mass.). VEGF, GAPDH, Sp1, and Sp4 band intensity values obtained by scanning the Polaroid on a Sharp JX-330 scanner (Sharp Electronics, Mahwah, N.J.); background signal is subtracted; and densitometric analysis is performed on the inverted image using Zero-D software (Scanalytics). Results are expressed as VEGF band intensity values normalized to GAPDH values and then by averaging three separate determinations for each treatment group. A similar approach will be used for VEGFR1, VEGFR2 and other angiogenic factors.

### EXAMPLE 9

### Plasmids and Constructs

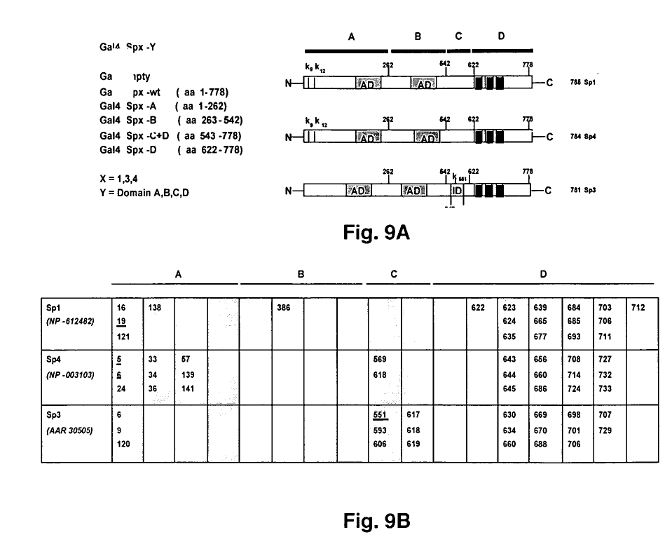

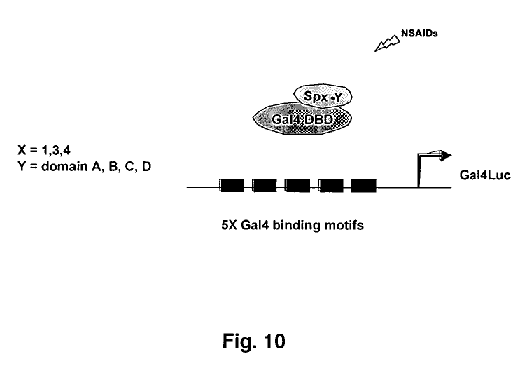

Gal4-Spx-y construct for Sp3 are generated using similar cloning technique that is used for Sp1 and Sp4 (FIG. 9A-9B and FIG. 10). Plasmids expressing the Gal4 DNA binding domain (amino acids 1-147) fused to Sp3 wildtype (pGAL4 Sp3 wildtype) and to domain A (pGAL4 Sp3 A) are provided by Dr. Grace Gill (Harvard Medical School, Boston, Mass.). pGAL4 Sp1 B, CD, and D are prepared as follows: pGAL4-Sp3B (aa 263-542) fragments is PCR amplified using the primer set of 5′ TCC GTC GAC GCA ACA GCG TTT CTG CAG CTA CC 3′ (sense) and 5′ TAT CTA GAA TCA GCC TTG AAT TGG GTG CAC CTG 3′ (antisense); the fragment is digested with SalI and XbaI, and finally cloned into the pM construct. pGAL4-Sp3CD (aa 543-778) and pMGAL4-Sp3D (aa 622-778) is generated using PCR. The primer sets for pGAL4-Sp3CD are 5′ TCC GGA TCC GCC TGC CGT TGG CTA TAG CAA AT 3′ (sense) and 5′ GTA TGT CGA CAT CAG AAG CCA TTG CCA CTG ATA TT 3′ (antisense); and primer sets for pGAL4 Sp3D (aa 635-788) are 5′ TCC GGA TCC GCC TGC CGT TGG CTA TAG CAA AT 3′ (sense) and the same antisense primer above. The PCR products are digested with BamHI and SalI and cloned into pM (Gal4-DBD) constructs. Mutations of the selected K residue are generated by site directed mutagenesis method and are verified by sequencing. Transfection of constructs into pancreatic cancer cells and preparation of nuclear extracts are described above.

### EXAMPLE 10

### Ubiquitinated Sp Proteins Immunoprecipitation

Panc1 and L3.6p1 cells are seeded into 150-mm tissue culture plates in maintenance medium and allowed to grow to approximately 90% confluence. Cells are then treated with DMSO, 50 μM of tolfenamic acid or a more active NSAID for 7 hr. Whole cell extracts for each treatment group are obtained using radioimmunoprecipitation assay (RIPA) buffer (50 mM Tris-HCL, 1% NP-40, 0.25% Na-deoxycholate, 150 mM NaCl, 1 mM EDTA) with the addition of protease inhibitor cocktail. Duplicate aliquots of 500 μg are used for the experiments. Cell extracts are diluted in ice-cold PBS containing protease inhibitor cocktail to a final volume of 1 ml, followed by the addition of 30 μL of protein A/G PLUS-agarose beads (Santa Cruz). The reactions are placed on a rocker at 4° C. for 3 hr, followed by centrifugation at 600 g at 4° C. for 5 min. A 900 μL aliquot of supernatant from each sample is transferred into a new Eppitube on ice. Rabbit polyclonal anti-Sp1 (1 μg), Sp4 (1 μg), Sp3 (1 μg), or normal rabbit IgG (1 μg) is added to the treatment groups, followed by addition of 30 μL of protein A/G PLUS-agarose beads. Samples are then placed on a rocker at 4° C. for 12 hr, followed by centrifugation at 600 g at 4° C. for 5 min. The supernatant is removed by aspiration and immunoprecipitates are washed with two cycles of 1 ml of ice-cold RIPA buffer followed by 1 ml of ice-cold PBS using centrifugation at 600×g at 4° C. for 5 min. The agarose pellet is resuspended in 50 μL of loading buffer, boiled, and centrifuged. The supernatant is separated by SDS-10% PAGE, electrophoresed to PVDF membrane. The PVDF membrane is probed with ubiquitin antibody (P4D1), then stripped and reprobed with Sp1 or Sp3 antibodies. The same membrane is stripped and reprobed with Sp4 antibody and visualized by ECL as described (17).

### EXAMPLE 11

### SUMOylated Sp Proteins Immunoprecipitation

Panc1 and L3.6p1 cells are seeded and treated as described for the ubiquitinated Sp protein immunoprecipitation experiment except that extract is pretreated with the cysteine protease inhibitor N-ethylmaleimide (NEM) to inhibit de-SUMOylating enzyme. In addition, the PVDF membrane is probed with SUMO-1 antibody first, then stripped and reprobed with Sp1 or Sp3 antibodies. The same membrane is then stripped and reprobed with Sp4 antibody and visualized by ECL as described above.

### EXAMPLE 12

### Glycosylated Sp Proteins Immunoprecipitation

Panc1 and L3.6p1 cells are seeded and treated as described for the in the ubiquitinated Sp protein immunoprecipitation experiments, except that the PVDF membrane is probed with the monosaccharide O-GlcNAc -specific RL-2 antibody first, then stripped and reprobed with Sp1 or Sp3 antibodies. The same membrane is then stripped and reprobed with Sp4 antibody and visualized by ECL as described above.

### EXAMPLE 13

### Identification of NSAIDs that Downregulated Sp1, Sp3 and Sp4 Expression in Pancreatic Cancer Cells

RNA interference studies have reported that VEGF is regulated by Sp1, Sp3 and Sp4 proteins in pancreatic cancer cells and celecoxib decreased Sp1 and VEGF expression in both in vitro and in vivo models (1, 8) Most studies on COX-1/2 inhibitors have shown growth inhibition at concentrations between 25-100 μM. Table 1 summarizes the list of individual NSAIDs.

TABLE 1
Summary of NSAIDs

| CLASS | COMPOUNDS |
|---|---|
| Salicylates | Aspirin, diflunisal, fendosal |
| Acetic acids | Indomethacin, acemetacin, anmetacin, sulindac, |
| | tolmetin, zomepirac, diclofenac, fenclofenac, |
| | isoxepac |
| Propionic acids | Ibuprofen, naproxen, betoprofen, fenoprofen, |
| | flurbiprofen, indoprofen, pirprofen, carprofen, |
| | fenbufen |
| Fenmates | Flufenamic acid, tolfenamic acid, mefenamic acid, |
| | meclofenamate, clonixin, flunixin, aceclofenac, |
| | niflumic acid |
| Pyrazoles | Febrazine, phenylbutazone, apazone, trimethazone, |
| | mofebutazone, bebuzone, suxibuxone |
| Osicams | Ampiroxicam, piroxicam, isoxicam, fenoxicam |

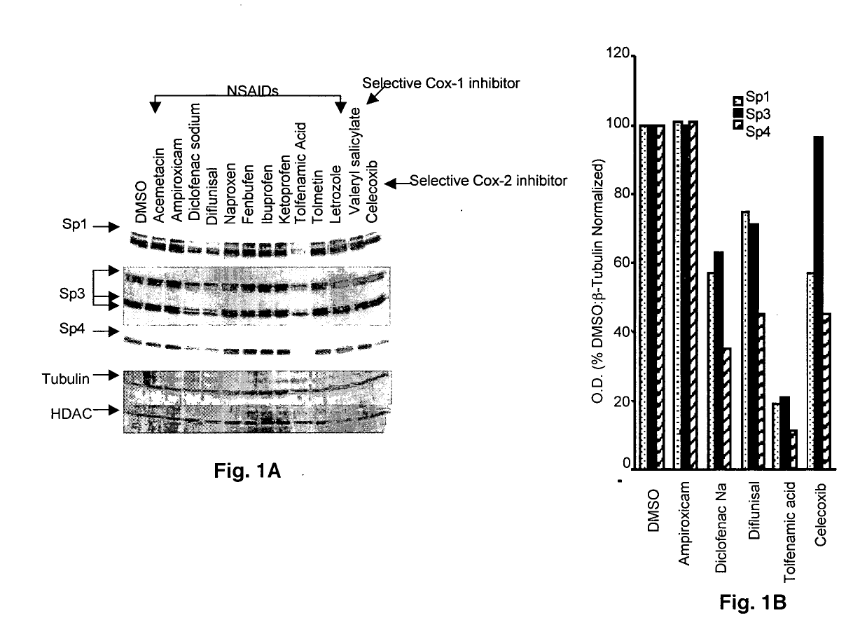

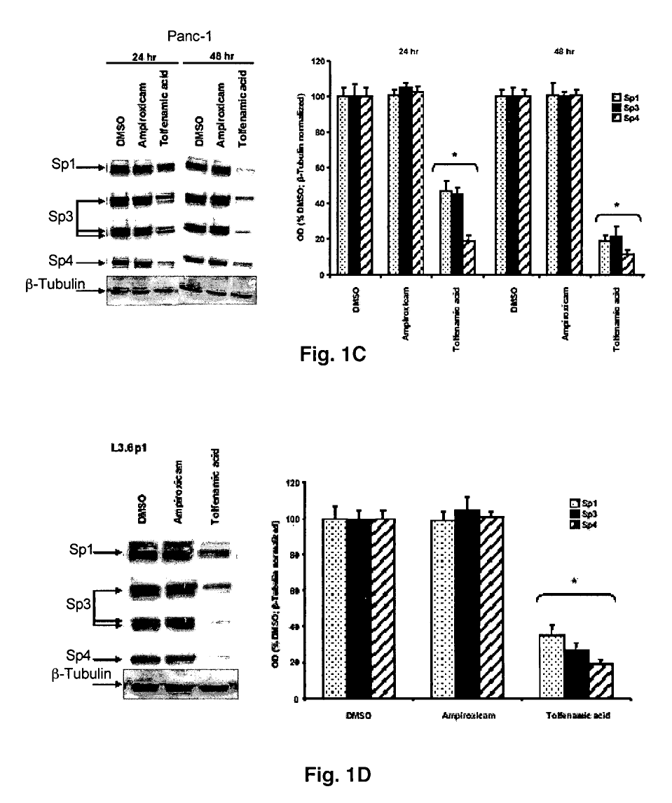

Hence, the present invention screened for the effects of 50 and 100 μM concentrations of different structural classes of nonsteroidal antiinflammatory drugs, celecoxib (COX-2 inhibitor) and valeryl salicylate (COX-1 inhibitor) on Sp1, Sp3 and Sp4 protein expression in Panc-1 cells after treatment for 48 hr. For the concentration of 50 μM (FIG. 1A), it was observed that celecoxib decreased the levels of Sp1 and Sp4 as previously described for colon cancer cells (2), whereas treatment with acemetacin, ampiroxicam, naproxen, fenbuten, ibuprofen, tolmetin, letrozole or valeryl salicylate had no effect on the levels of these proteins. In distinct contrast, the diphenyl and diphenylamine-derived carboxylic acid compounds decreased expression of Sp1, Sp3 and Sp4 proteins in Panc-1 cells and their order of potency was tolfenamic acid>diclofenac˜diflunisal (FIG. 1B). Similar results were obtained using 100 μM concentration of nonsteroidal antiinflammatory drugs.

Additionally, 50 μM tolfenamic acid induced a time-dependent decrease in Sp1, Sp3 and Sp4 proteins in Panc-1 cells (FIG. 1C) with a >80% decrease in levels of all three proteins after treatment for 48 hr. In distinct contrast, treatment with ampiroxicam (a negative control) did not affect Sp protein levels compared to those in solvent (DMSO)-treated cells. The comparative effects of 50 μM tolfenamic acid and ampiroxicam on sp protein expression in the highly metastatic L3.6p1 pancreatic cancer cell line (FIG. 1D) were similar to those observed in Panc-1 cells (FIG. 1C) and >65% decrease of Sp1, Sp3 and Sp4 proteins was observed after treatment for 48 hr.

Both dose (25-150 μM)- and time (12-76 hr)-dependent decreases in Sp1, Sp3 and Sp4 in pancreatic cancer cell lines, and intracellular decreases is confirmed by immunocytochemistry. Nuclear extracts are incubate with a [³²P]GC (consensus GC-rich oligonucleotide and analyzed in gel mobility shift assays (±Sp antibodies) as described (17, 18).

### EXAMPLE 14

### Tolfenamic Acid Decreased Transactivation in Pancreatic Cancer Cells Transfected with VEGF Promoter Constructs

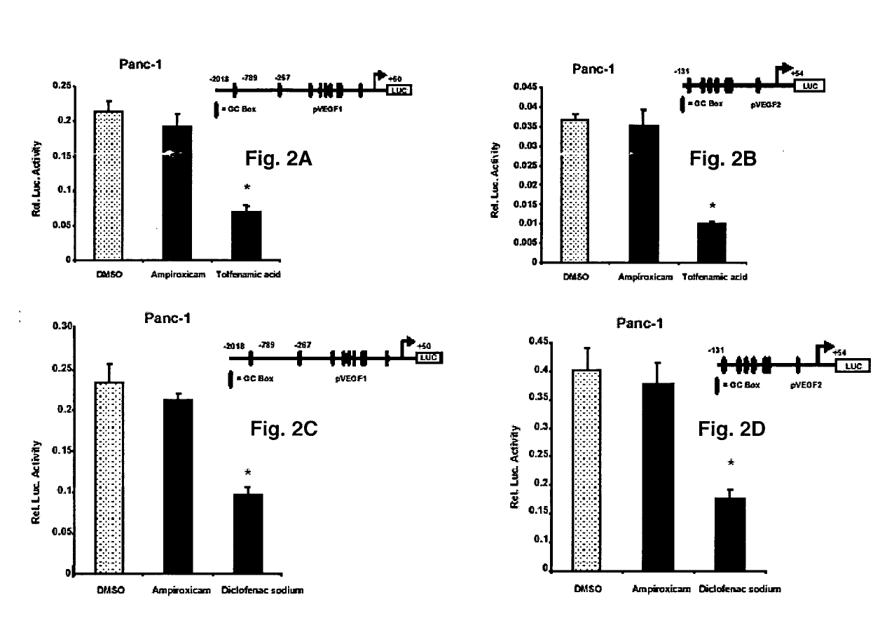

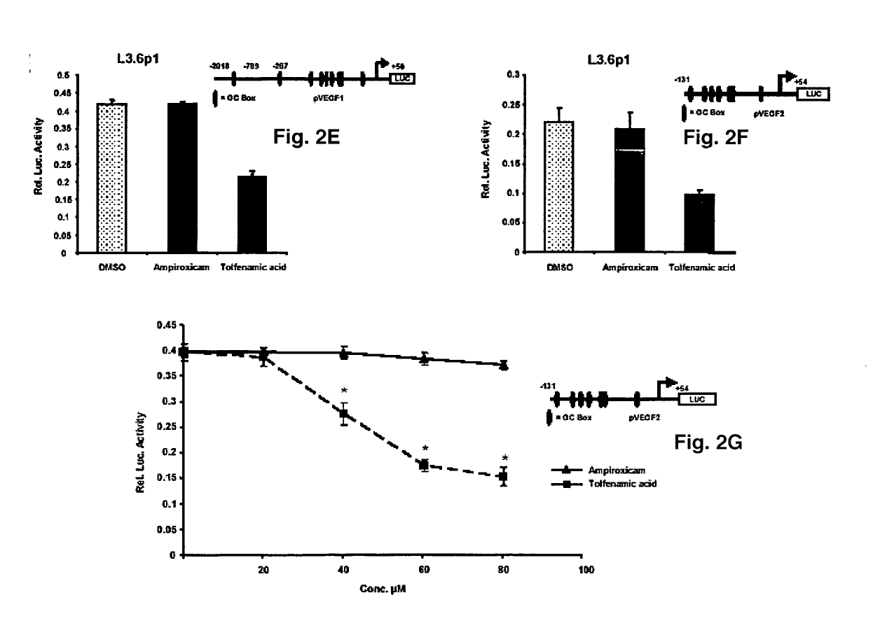

Previous studies showed that COX-2 inhibitors or small inhibitory RNAs for Sp proteins decreased VEGF expression in colon and pancreatic cells (1, 2, 6 and 8). The effects of DMSO, 50 μM tolfenamic acid and 50 μM ampiroxicam on VEGF expression were initially investigated in Panc-1 cells transfected with the pVEGF1 and pVEGF2 constructs which contain −2018 to +50 and −131 to +54 VEGF promoter inserts, respectively (FIGS. 2A and 2B). Tolfenamic acid but not ampiroxicam significantly decreased activities in cells transfected with both constructs.

In a parallel series of experiments in Panc-1 cells, it was shown that diclofenac sodium which induces Sp protein degradation (FIG. 1A) also decreased transactivation in Panc-1 cells transfected with pVEGF1 (FIG. 2C) and pVEGF2 constructs (FIG. 2D). Decreased luciferase activity after treatment with tolfenamic acid and diclofenac sodium was consistent with previous studies that showed that activation of these constructs was dependent on interactions of Sp1, Sp3 and Sp4 proteins with proximal GC-rich motifs (1, 2, 6 and 8).

Additionally, a similar experiment was performed in L3.6p1 cells (FIGS. 2E and 2F) and the results similar to those observed in Panc-1 cells were obtained. These data were consistent with the observed downregulation of Sp1, Sp3 and Sp4 protein in L3.6p1 and Panc-1 cells treated with tolfenamic acid (FIG. 1). Furthermore, a concentration-dependent decrease in transactivation was observed in Panc-1 cells transfected with pVEGF2 and treated with 20-80 μM of tolfenamic acid (FIG. 2G), whereas ampiroxicam showed no effect. Similar results were obtained for the COX-1 inhibitor valeryl salicylate (data not shown).

### EXAMPLE 15

### Tolfenamic Acid Decreased Sp Protein Binding to GC-rich VEGF Promoter Oligonucleotides

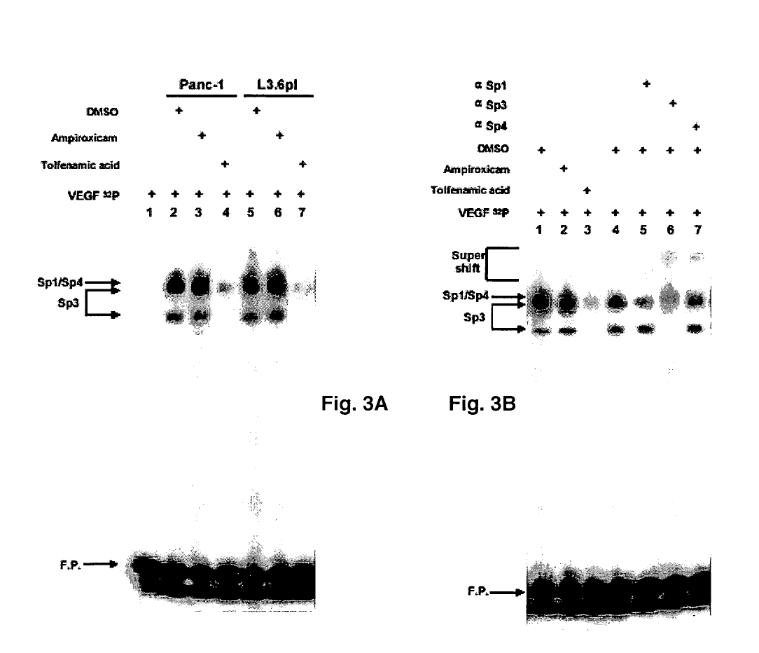

VEGF is regulated by Sp proteins binding to proximal GC-rich promoter sequences (1, 2, 6 and 8) Hence, the effects of tolfenamic acid and ampiroxicam on DNA binding was examined using mobility shift assay (FIGS. 3A and 3B). Panc1 or L3.6p1 cells were treated with DMSC, 50 μM tolfenamic acid or 50 μM ampiroxicam for 48 hr. Nuclear extracts from these cells were isolated, incubated with VEGF³²P and anlyzed in gel mobility shift assays.

The radiolabeled oligonucleotide contained a GC-rich region (−66 to −47) from the VEGF promoter that binds Sp proteins. Sp3 and Sp1/Sp4 (overlapping) retarded bands of similar intensity were observed after incubation with extracts from Panc-1 cells treated with DMSO or ampiroxicam (lanes 2 and 3) or comparable extracts from L3.6p1 cells (lanes 5 and 6). In distinct contrast, these retarded bands were decreased using extracts from Panc-1 (lane 4) or L3.6p1 (lane 7) cells treated with tolfenamic acid. Retarded bands were not observed using the VEGF³²P alone (lane 1).

Additionally, results in FIG. 3B also illustrate the pattern of binding with extracts from cells treated with DMSO, ampiroxicam and tolfenamic acid (lanes 1-3, respectively). The retarded bands formed in extracts from solvent (DMSO)-treated cells (lane 4) exhibited supershifted bands after coincubation with antibodies for Sp1 (lane 5), SP3 (lane 6) and Sp4 (lane 7). The supershifted complexes are indicated by arrows. The results discussed herein demonstrated that tolfenamic acid decreased Sp1/Sp3/Sp4 binding to GC-richoligonucleotides identical with the −66 to −47 region of the VEGF promoter and this was consistent with decreased transactivation observed in transient transfection studies.

### EXAMPLE 16

### NSAID-induced Antiangiogenic Activity

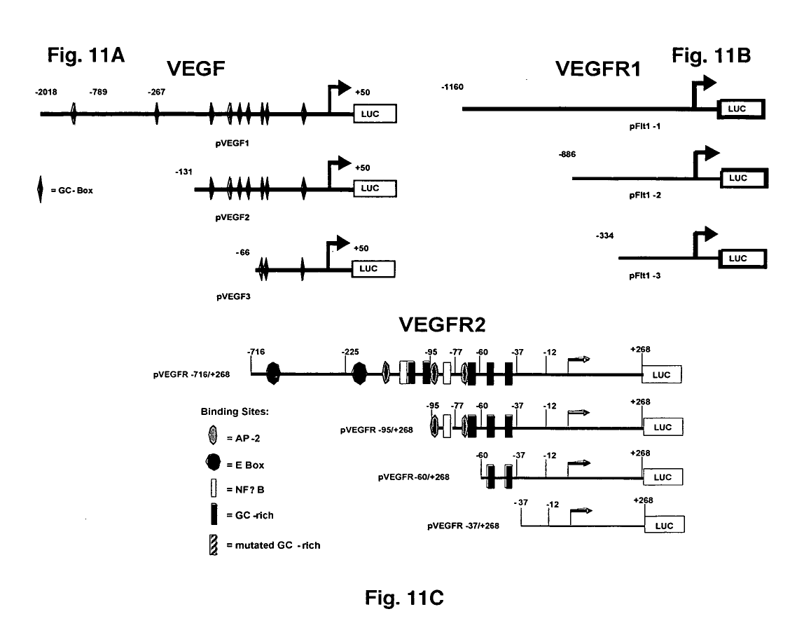

Using active NSAIDs, the time- and dose-dependent decrease in VEGF, VEGFR2 and VEGFR1 mRNAs/proteins is determined by Western blot, and RT-PCR and immunocytochemistry is performed to confirm downregulation of these angiogenic factors. NSAID-induced decreases in VEGF/VEGFR1/VEGFR2 is also be confirmed in transient transfection studies using constructs containing promoter inserts (FIG. 11). These results correlate a critical response associated with downregulation of Sp proteins, namely downregulation of angiogenic factors that are regulated by Sp transcription factors.

Confirmation that tolfenamic acid decreased VEGFR1 expression in Panc-1 and L3.6p1 cells was determined by immunohistochemical analysis (FIG. 15). In Panc-1 cells, VEGFR1 immunostaining was observed in cells treated with DMSO or ampiroxicam, whereas tolfenamic acid decreased VEGFR1 staining. VEGFR1 expression was lower in L3.6p1 cells; however, the pattern of treatment related effects were comparable in both Panc-1 and L3.6p1 cells.

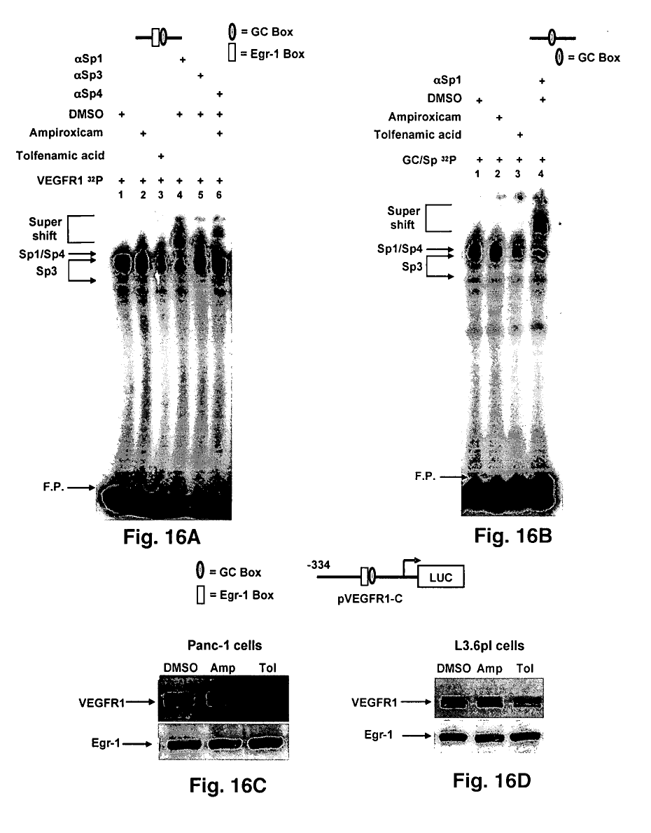

The proximal region of the VEGFR1 promoter contains GC-rich and an Egr-1 sites, and the effects of tolfenamic acid on VEGFR1 expression through degradation of Sp proteins cannot exclude a role for Egr-1 in this response. FIGS. 16A and 16B compare the gel mobility shift and antibody supershift patterns of nuclear extracts from Panc-1 cells bound to the proximal region of the VEGFR1 promoter containing both GC-rich and Egr-1 sites (VEGFR1³²P) or GC-rich sites alone (GC/SP³²P). Extracts from cells treated with DMSO, ampiroxicam and tolfenamic acid gave similar Sp1, Sp3 and Sp4-DNA retarded bands using both radiolabeled oligonucleotides (FIGS. 16A and 16B, lanes 1-3); however, the retarded band intensity was markedly decreased using extracts from cells treated with tolfenamic acid. Antibodies against Sp1 (FIGS. 16A and 16B, lane 4) supershifted the large Sp1-DNA retarded band and both Sp3 and Sp4 antibodies also induced formation of supershifted complexes (FIG. 16A, lanes 5 and 6). These results show that the presence or absence of the Egr-1 site did not affect Sp protein interactions with the VEGFR1 promoter. Moreover, Western blot analysis of whole cell lysates from Panc-1 and L3.6p1 cells after treatment with DMSO, ampiroxicam or tolfenamic acid showed that only the latter compound decreased VEGFR1 protein levels, whereas Egr-1 protein was unchanged in all treatment groups (FIGS. 16C and 16D).

Since Sp proteins regulate expression of VEGFR1, the effects of tolfenamic acid on luciferase activity were further investigated in cells transfected with pVEGFR1A, pVEGFR1B and pVEGFR1C, and on VEGFR1 mRNA levels. Tolfenamic acid but not DMSO or ampiroxicam decreased luciferase activity in Panc-1 cells transfected with pVEGFR1A, pVEGFR1B and pVEGFR1C (FIGS. 17A-17C) and tolfenamic acid also decreased VEGFR1 mRNA levels in Panc-1 and L3.6p1 cells (FIG. 17D).

Previous studies have reported that activation of VEGFR1 by VEGF-B in pancreatic cancer cells results in enhanced phosphorylation of MAPK and increased cell migration and invasio. The role of Sp proteins in mediating these responses was therefore investigated by determining the effects of tolfenamic acid on activation of MAPK by VEGF-B. FIGS. 18A and 18B illustrate that after treatment of Panc-1 and L3.6p1 cells, respectively, with VEGF-B for 5 or 10 min, there was increased phosphorylation of MAPK1/2 in cells pretreated with DMSO or 50 μM ampiroxicam for 36 hr, and VEGFR1 and total MAPK1/2 levels were unchanged. In contrast, pretreatment with 50 μM tolfenamic acid decreased VEGFR1 expression and this was paralleled by decreased phospho-MARK1/2, whereas total MAPK protein levels were not affected. Thus, inhibition of VEGF-B/VEGFR1 signaling by tolfenamic acid was related to decreased VEGFR1 through degradation of Sp proteins and this inhibitory response was similar to that observed in a previous study using neutralizing VEGFR1 antibodies. The importance of Sp protein in VEGFR1-dependent Panc-1 cell migration was determined using a cell migration assay on collagen IV-coated plates. The results show that VEGF-B induces Panc-1 cell migration in all treatment groups (FIG. 18C); however, in cells treated with DMSO (set at 100%), 50 μM ampiroxicam, or 50 μM tolfenamic acid cell migration which was observed in the absence of VEGF-B was only significantly inhibited by tolfenamic acid (FIG. 18C). VEGF-B enhanced cell migration in this assay and, in cells cotreated with VEGF-B plus tolfenamic acid, the latter compound significantly inhibited VEGF-B-induced cell migration.

Previous studies using an orthotopic model for pancreatic cancer (using L3.6p1 cells) showed that tolfenamic acid (50 mg/kg) but not gemcitabine decreased Sp proteins, tumor growth, and angiogenesis, and tumor tissue from these animals was also stained for VEGFR1 (FIG. 18D). The results show decreased VEGFR1 expression only in tumors from tolfenamic acid-treated mice. These results demonstrate that Sp proteins regulate VEGFR1-mediated responses including cell migration in pancreatic cancer cells indicating that agents such as tolfenamic acid that target Sp proteins (for degradation) are an important new class of mechanism-based antiangiogenic compounds that decrease Sp-dependent expression of VEGF, VEGFR2 and VEGFR1.

### EXAMPLE 17

### Role of Proteasome Pathway in Sp Protein Degradation

The role of the proteasome pathway in mediating degradation of Sp proteins is investigated. Posttranslational modification of Sp proteins are summarized in Table 2 (phosphorylation, sumoylation and ubiquitination) can markedly affect their expression/activity in cells, and the effects of tolfenamic acid on these modifications of Sp proteins will be investigated in Panc1 and L3.6p1 cells.

TABLE 2
Post-translational modification of Sp proteins.*

| Post- | | | |
|---|---|---|---|
| translational | Sp1 | Sp3 | Sp4 |
| modification | (AA and Domain) | (AA and Domain) | (AA and Domain) |
| Glycosylation | Ser/Thr residues | Not determined | Not determined |
| | Activation domain | | |
| Phosphorylation | Ser/Thr residues, | Not determined | Ser/Thr residues |
| | some are also | | Activation |
| | targeted by | | domain and DNA- |
| | glycosylation | | binding domain |
| | Activation | | |
| | domain and DNA- | | |
| | binding domain | | |
| Acetylation | Lysin | Inhibitory | Not determined |
| | DNA-binding | domain | |
| | domain | (IKEE motif) | |
| | | DNA binding | |
| | | domain | |
| Sumoylation | Not determined | Inhibitory | Not determined |
| | | domain | |
| | | (IKEE motif- | |
| | | Lysine 551) | |
| Ubiquitination | Lysine | Not detected | Lysine |
| | N-terminal and other | | N-terminal and |
| | domains | | other domains |

*The various domains of Sp proteins

Tolfenamic acid and/or two active NSAIDs that target Sp proteins for degradation are to investigate the mechanisms of Sp1, Sp3 and Sp4 proteins degradation and the role of the proteasome pathway in this process. Pancreatic cancer cells are cotreated with NSAIDs and several proteasome and proteasome/protease inhibitors for 36-48 hr, and Sp protein levels are analyzed by Western blot analysis and immunohistochemistry.

Inhibition of Sp protein degradation by tolfenamic acid and related compounds is quantified relative to the control treatment (DMSO) and the structural protein (β-tubulin). The peptide aldehydes (such as N-acetyl-L-leucinyl-L-leucinal-L-norleucinal, LLnL, and N-carbobenzoxyl-L-leucinyl-L-leucinyl-L-norvalinal, MG115) are used and these peptides strongly inhibit peptidase activities associated with the proteasome complex (25). However, these peptide aldehydes can also inhibit the cysteine proteases found in lysosomes and calpains (25). Therefore, more selective proteasome inhibitors are used to definitively establish a major role for proteasomes in Sp protein degradation by NSAIDs.

Lactacystin is a chemically distinct proteasome inhibitor that reversibly inhibits the post-acidic activity of purified proteasomes through formation of clasto-lactacystin β-lactone, which reacts with the N-terminal threonine of subunit X (26, 27). Lactacystin and gliotoxin are used as proteasome inhibitors to confirm that Sp proteins are degraded by proteasomes.

### EXAMPLE 18

### Identification of Structural Domains that are Required for Sp 1 Sp3 and Sp4 Protein Degradation by Tolfenamic Acid and Related NSAIDs

The proteasome complex recognizes its specific targets in several different pathways and these are related, in part, to modification of critical lysine residues with ubiquitin or ubiquitin-like peptides that are recognized by the proteasome complex (see below). FIG. 9A-9B summarizes the domain structures of Sp1, Sp3 and Sp4, and this contains activation domains (AD), inhibitory domains (ID), and the characteristic C-terminal zinc finger motifs required for DNA binding. The present invention has prepared Gal4-Sp1/Gal4-Sp3/Gal4-Sp4 constructs, and the truncated forms contain the C-terminal A, N-terminal D, and internal C and B domains of Sp1 and Sp3 fused to the yeast Gal4 DBD (aa 1-147). The corresponding cDNAs encoding various fragments of Sp1/Sp4 are generated by restriction enzyme digested and PCR amplification using high fidelity Pfu DNA polymerase and cloned into the Gal4 DBD vector; the resulting plasmids [Gal4/Sp3(A-D) fragments] are used along with the corresponding wild-type/mutant GAL4-Sp1/Gal4-Sp4 in studies designed to determine which domains of Sp proteins are targeted for degradation.

Plasmid constructs that express chimeric proteins containing the DBD of the yeast Gal4 protein fused to different domains of Sp proteins (Gal4-Sp) (FIG. 10) are transfected into Panc1 and L3.6p1 cells in 6-well plates along with 500 ng of a reporter plasmid (Gal4-Luc) containing 5 tandem Gal4 response elements linked to bacterial luciferase. Cells are then treated with different concentrations of NSAIDs (15-100 μM). Decreased responses are indicative of NSAIDs-induced degradation of specific domains of Sp1, Sp3 and Sp4, and thereby identify specific domains susceptible to proteasome degradation. That decreased transactivation is linked to activation of the proteasome pathway by NSAIDs is confirmed using the proteasome inhibitors that will block the decreased transactivation in transfected cells.

Previous studies have demonstrated that NSAIDs (COX-2 inhibitors) induce proteasome-dependent degradation of Sp1 and Sp4 in colon cancer cells, and this is associated with increased ubiquitination of both proteins (19). The present invention uses pancreatic cancer cells which are treated with different concentrations of NSAIDs for 24 hr, and whole cell lysates are immunoprecipitated with IgG or antibodies to Sp1, Sp3 or Sp4 followed by Western blot analysis for Sp proteins and for ubiquitinated proteins. These studies confirm that NSAIDs induce ubiquitination of wild-type (endogenous) Sp1 and Sp4 in pancreatic cancer cells as previously observed in colon cancer cells (19). These same approaches are used for the wild-type/mutant Gal4-Sp constructs in which the chimeric proteins is immunoprecipitated with the Gal4 antibodies and analyzed by SDS-PAGE/Western blots with ubiquitin antibodies.

In studies in colon cancer cells, COX-2 inhibitors did not induce degradation (or ubiquitination) of Sp3 and, in the present invention, ubiquitination of Sp3 is also investigated. However, Sp3 is expressed as four isoforms that become post-translationally modified by SUMOylation (18) in which Sp3 is conjugated with small ubiquitin-related modifier (SUMO) (29-34). A consensus SUMO acceptor site consisting of the sequence ψKXE has been identified where ψ is a large hydrophobic amino acid and K (lysine) is the site of SUMO conjugation and SUMO modification of Sp3 is specific at lysine residue 551. Therefore, the same approach is used for detecting SUMOylation of Sp3, Sp1 and Sp4 by immunoprecipitation/SDS-PAGE and Western blot analysis as described for ubiquitination of wild-type/variant Sp1 and Gal4-Sp proteins. FIG. 10 also summarizes the lysine sites in Sp proteins including lysine residues at amino acids 16 and 19 (for Sp1) and 5 and 6 (for Sp4) and, for Sp1, these amino acids are known to be important for proteasome degradation. Lysine residues within domains of Sp1, Sp3 and Sp4 that undergo NSAID-induced proteasome degradation are mutated to alanines and their degradation is then investigated in pancreatic cancer cells as described for the wild-type constructs. The lysine mutation studies confirms the role of specific lysines in Sp proteins in mediating their NSAID-induced degradation.

### EXAMPLE 19

### Post-translational Modifications of Sp Proteins Induced by Tolfenamic Acid

Sp proteins are also subjected to other post-transcriptional modifications (35) including phosphorylation, acetylation and glycosylation (36-39). Phosphorylation and acetylation of Sp proteins can modify their activities as transcription factors but does not affect proteins expression (40-44). In contrast, glycosylation has been associated with protein stability and susceptibility to degradation (45-47). This modification involves the covalent linkage of the monosaccharide O-GlcNAc to serine or threonine residues and Sp 1 protein was first transcription factor known to contain this modification (47). Hypoglycosylated Sp1 is more susceptible to proteasome-dependent degradation and each molecule of Sp1 contains an average of eight O-Glc- NAc modifications (47) and one of these sites is in the transcriptional activation domain of the molecule. To determine if the glycosylation states of Sp1, Sp3 and Sp4 are affected by treatment with NSAIDs, glucosamine which is used primarily as a substrate for protein glycosylation is used to test whether it can block Sp protein hyopglycosylation and degradation by NSAIDs. Glucosamine treatment produces higher molecular weight hyperglycoslated forms of Sp1 protein and block its degradation under glucose starvation condition (47). Panc1 and L3.6p1 cells treated with 5 mM glucosamine and/or different concentrations of NSAIDs for 24 and 48 hr, are analyzed for Sp1, Sp3 and Sp4 proteins levels by Western blotting. To investigate the glycosylation state of Sp1/Sp3/Sp4 proteins after NSAID treatment, cells are cotreated with 3 μM lactacystin to prevent loss of Sp proteins by NSAIDs, then Sp proteins are immunoprecipitated by Sp specific antibodies and analyzed for glycosylation by Western blot using RL-2 epitope GlcNAc-specific antibody.

### EXAMPLE 20

### Tolfenamic Acid Decreased VEGF mRNA and Protein Expression and Activated Proteosome-dependent Degradation of Sp1, Sp3 and Sp4

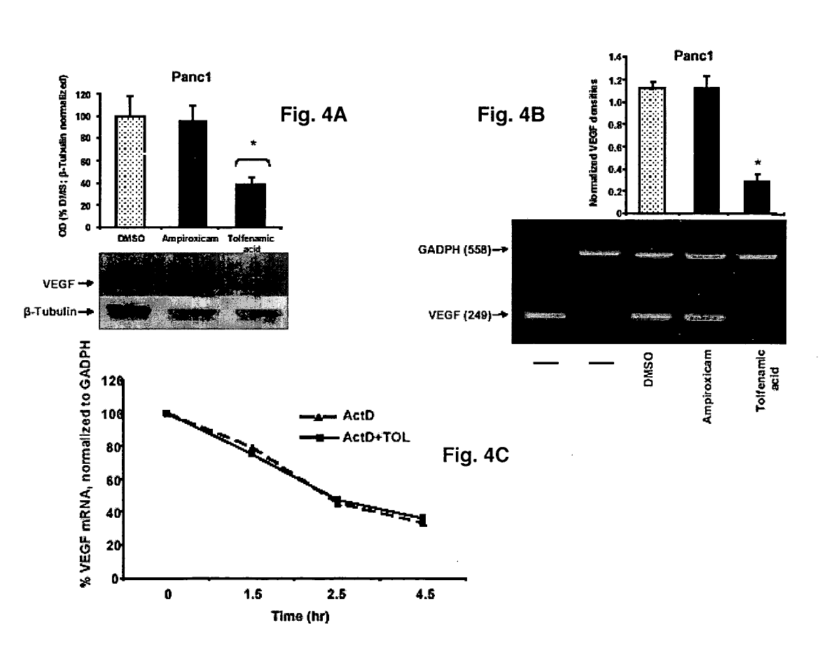

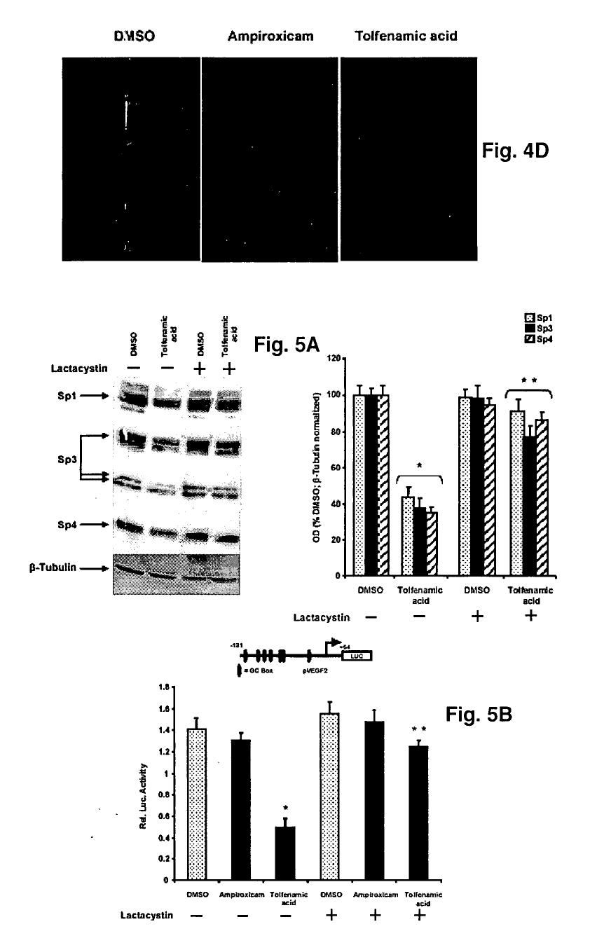

Panc-1 cells were treated with DMSO, 50 μM ampiroxicam or teolfenamic acid for 48 hr and VEGF protein levels (relative to β-tubulin) were analyzed (FIG. 4A). The results showed that tolfenamic acid decreased VEGF expression by >60% compared to solvent (DMSO) control. The effects of tolfenamic acid on VEGF mRNA levels were also investigated in Panc-1 cells by RT-PCR and compared to levels in solvent (DMSO) and ampiroxicam-treated cells.

Tolfenamic acid treatment for 24 hours decreased VEGF mRNA levels by >60% (compared to DMSO) (FIG. 4B). However, this decrease was not due to decreased message stability since VEGF mRNA levels decreased at similar rates in Panc-1 cells treated with actinomycin D alone or in combination with 50 μM tolfenamic acid. In Panc-1 cells, VEGF protein was secreted and immunostaining with VEGF antibodies showed a significant lawn of extracellular VEGF staining in cells treated with DMSO or ampiroxicam (FIG. 4D). In distinct contrast, after treatment of Panc-1 cells for 48 hr with 50 μM tolfenamic acid, the immunostaining of secreted VEGF protein was almost non-detectable. These results clearly demonstrated that tolfenamic acid-dependent downregulation of Sp1, Sp3 and Sp4 protein expression resulted in decreased levels of VEGF mRNA and protein which was consistent with the reported Sp-dependent regulation of VEGF (1, 2, 6 and 8).

Furthermore, it was also reported that the COX-2 inhibitor nimesulide decreased levels of Sp1 and Sp4 (but not Sp3) in colon cancer cells through activation of the proteasome pathway and this response was blocked by the proteasome inhibitor gliotoxin (2). The results in FIG. 5A demonstrated that tolfenamic acid decreased Sp1, Sp3 and Sp4 proteins and differed from nimesulfide (2) and celecoxib (FIG. 1A) which induced degradation of Sp1 and Sp4 but not Sp3. However, in Panc-1 cells co-treated with 50 μM tolfenamic acid plus 2 μM of proteosome inhibitor lactacystin, there was a significant inhibition of Sp1, Sp3 and Sp4 protein degradation. Thus, like COX-2 inhibitors (celecoxib and nimesulide), tolfenamic acid activated proteasome-dependent degradation of Sp1 and Sp4 in Panc-1 cells. Although nonsteroidal antiinflammatory drugs induced degradation of Sp3, COX-2 inhibitors had no effect on Sp3. Additionally, decreased transactivation in Panc-1 cells transfected with pVEGF2 and treated with tolfenamic acid was also blocked in cells co-treated with lactacystin (FIG. 5B) and similar results were obtained for gliotoxin. Ampiroxicam did not affect luciferase activity in presence or absence of lactacystin. These results also confirmed the role of Sp proteins as key mediators of VEGF expression.

### EXAMPLE 21

### Tolfenamic Acid Inhibited Growth of Pancreatic Cancer Cells

Although it was reported that nonsteroidal antiinflammatory drugs and COX-2 inhibitors inhibited the growth of pancreatic cancer cells, the underlying mechanisms were not well understood. Sp proteins play a key role in regulating genes involved in cancer cell growth and angiogenesis, their role in pancreatic cancer cell growth was investigated in cells treated with tolfenamic acid and other nonsteroidal antiinflammatory drugs.

To determine the growth inhibitory effects of the active-NSAIDs in pancreatic cancer cell cells, cell cycle phase distribution is determined by staining with propidium iodide and analysis by flow cytometry using the FACS Calibur Immunocytometry Systems benchtop flow cytometer combined with a Macintosh computer using CELL Quest software (17). Co-staining of the cells for direct incorporation of BrdU is carried out using direct immunofluorescence microscopy, anti-BrdU primary antibodies, and rhodamine-conjugated secondary antibodies.

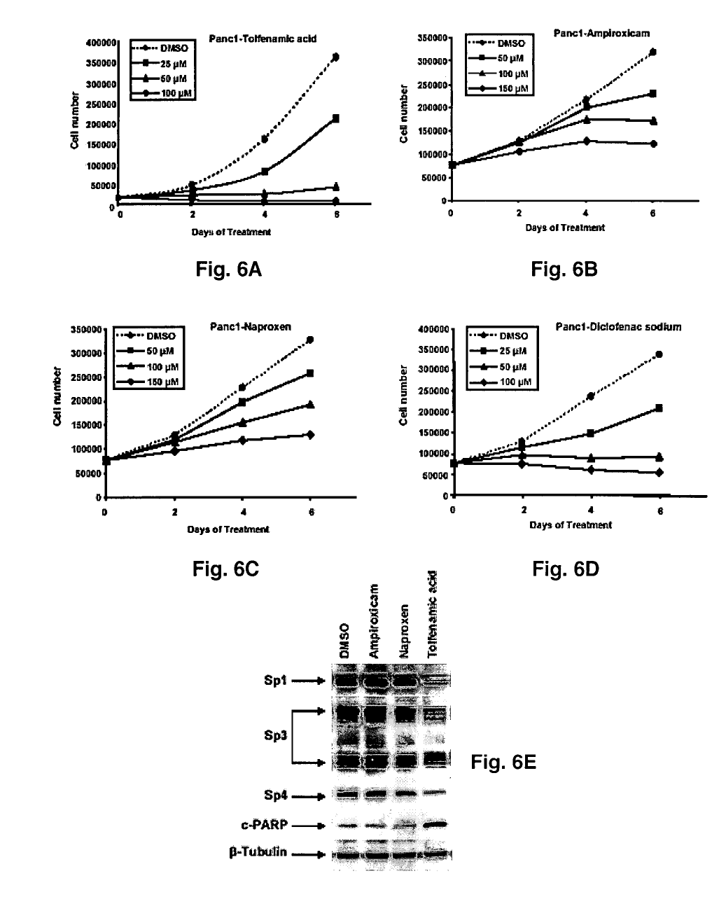

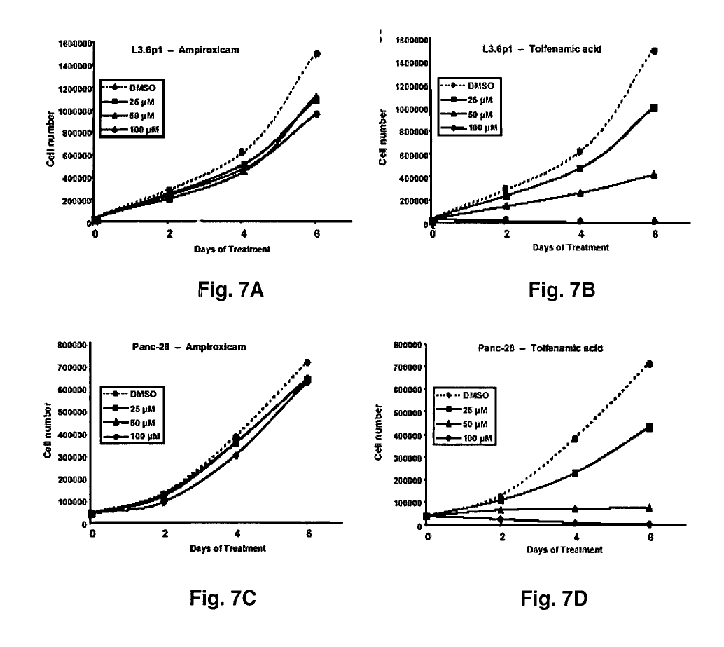

It was observed that ampiroxicam, naproxen, diclofenac sodium and tolfenamic acid decreased growth of Panc-1 cells and the latter two compounds also exhibited higher potencies in this assay (FIGS. 6A-6D). Since it was possible that the observed differences in the effects of 50 μM concentrations of nonsteroidal antiinflammatory drugs on Sp protein degradation (FIG. 1A) may be related in part to their relative growth inhibitory activities, the effects of tolfenamic acid, ampiroxicam and naproxen on Sp protein expression was compared using concentrations that induced comparable inhibition of Panc-1 cell proliferation. Thu, Panc-l cells were treated with 50 μM tolfenamic acid, 150 μM naproxen and 150 μM ampiroxicam for 48 hr and the expression of the proteins compared by Western blot. It was observed that only tolfenamic acid induced protein degradation and PARP cleavage. These data supported the results of the initial screening assay (FIG. 1A) showing that only nonsteroidal antiinflammatory drugs containing the diphenyl/diphenylamine carboxylic acid structure induced Sp protein degradation. Using a similar range of concentrations in L3.6p1 (FIGS. 7A and 7B) and Panc-28 (FIGS. 7C and 7D) cells, it was apparent that tolfenamic acid was also more potent as an inhibitor of pancreatic cell growth than ampiroxicam. These results suggested that the growth inhibitory effects of tolfenamic acid in pancreatic cells were associated in part, with degradation of Sp proteins.

### EXAMPLE 22

### Modulation of p27 and other cdki Proteins/genes

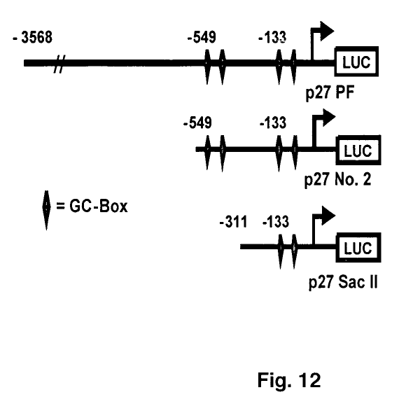

Recent studies show that Sp3 protein suppresses the cdk1, p27 and Sp3 knockdown by RNA interference, induces p27 expression, and inhibits G1→S phase progression (17). Three active NSAIDs are used and cells are treated as described above. Expression of p27 protein/mRNA is determined, and the effects on cell proliferation and the % distribution of cells in G₀/G₁, S and G₂/M phases of the cell cycle is determined by FACS analysis as described (17). In addition, the effects of NSAIDs on p27 promoter expression is examined using a series of GC-rich constructs (FIG. 12).

### EXAMPLE 23

### Orthotopic Implantation of L3.6p1 Cells and Treatment with NSAIDs Gemcitabine and their Combination

At least three active NSAIDs are administered at doses of 10, 25 and 50 mg/kg day and gemicitabine is used at doses of 50, 75 and 100 mg/kg/day. In combination treatments, a minimally effective dose of gemcitabine in combination with at least two doses of the NSAIDs is used to determine potential interactive effects on tumor growth liver metastasis and histopathology.

Male athymic nude mice (NCI-nu) are purchased from the Animal Production Area of the National Cancer Institute Frederick Cancer Research and Development Center, housed and maintained under specific pathogen-free conditions in accordance with current regulations and standards of the United States Department of Agriculture. Injection of L3.6p1 cells (with >90% viabild³ is performed as previously described (21). Seven days after implantation of tumor cells into the pancreas of each mouse, 5 mice are killed to confirm the presence of tumor lesions.

Mice (10 animals per treatment group) are randomized to receive one of the following treatments: (1) thrice weekly oral administrations of vehicle solution which will serve as a control group; (2) thrice weekly oral administrations of at least three NSAIDs at doses of 10, 25, and 50 mg/kg/day; (3) twice weekly i.p. injections of 50, 75 and 100 mg/kg/day gemeitabine alone; and (4) combinations of gemcitabine and NSAIDs. Mice are sacrificed on day 35 and body weights determined. Primary tumors in the pancreas are excised, measured, weighed. For IHC and H&E staining procedures, one part of the tumor tissue is fixed in formalin and embedded in paraffin, and another part is embedded in OCT compound, rapidly frozen in liquid nitrogen, and stored at −70° C. Visible liver metastases is counted with the aid of a dissecting microscope, and the tissues processed for H&E staining.

### EXAMPLE 24

### Immunohistochemical Determination of VEGF Sp Proteins and VEGFR2

Paraffin-embedded tissues are used for identification of Sp proteins, VEGFR2p27 and VEGF. Sections (4-6 μM thick) are mounted on positively charged Superfrost slides (Fischer Scientific, Co, Houston, Tex.) and dried overnight. Sections are deparaffinized in xylene and then treated with a graded series of alcohol [100, 95, and 80% ethanol (v/v) in double distilled H₂O] and rehydrated in PBS (pH 7.5). Tissues are treated with pepsin (Biomeda) for 15 min at 37° C. and washed with PBS. A positive reaction is visualized by incubating the slides with stable 3,3′-diaminobenzidine for 10-20 min. Sections are rinsed with distilled water, counterstained with Gill's hematoxylin for 1 min, and mounted with Universal Mount (Research Genetics). Control samples exposed to secondary antibody alone show no specific staining. Tumor sample lysates are also examined by Western blot analysis for the same proteins.

### EXAMPLE 25

### Sp Protein Knockdown by RNA Interference in vivo

Recent studies show that siRNAs and derivatized siRNA can be injected into mice and efficient knockdown of target proteins observed for extended time periods. The present invention uses the siSTABLE™ (Dharmacon) siRNAs, which are chemically modified siRNAs with 500 times greater stability in vivo due to their resistance to nuclease activity. These in vivo stable siRNA can provide long term knockdown of Sp1, Sp3 and Sp4 proteins. The siRNAs are administered by intraperitoneal injection (10-50 μg/mouse). Optimal concentration and dosing frequency for successful knock down of Sp1, Sp3, and Sp4 in the liver and pancreas of mice is determined. These concentrations are then used in the orthotopic model for pancreatic cancer and administered by intraperitoneal injection. The duration of the proposed treatment is for 4 weeks, and pancreatic tumors, liver metasasis, histopathology and immunohistorhemistry is carried out as described for the NSAIDs above. The treatment group size is decreased to 5 mice due to the limited availability of the siRNAs.

### EXAMPLE 26

### Tolfenamic Acid Inhibited Pancreatic Tumor Growth and Metastasis in an Orthotropic Model

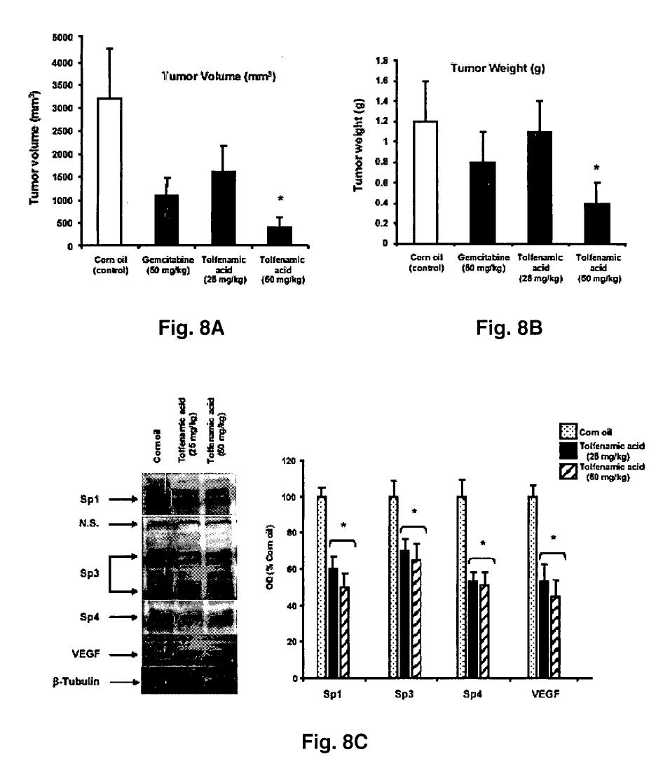

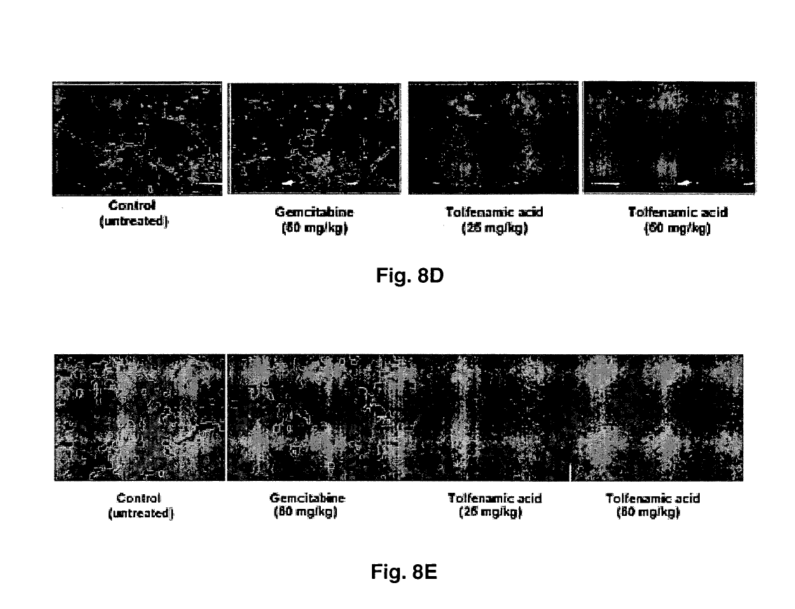

The potential antitumorigenic and antiangiogenic activity of tolfenamic acid against pancreatic tumors was investigated in an orthotropic athymic nude mouse model. L3.6p1 cells were used for this study based on their aggressive growth and production of liver metastases (3,4) Tolfenamic acid (25 or 50mg/kg/d) decreased median tumor weights and volumes (FIGS. 8A and 8B) and these treatments also decreased the percent incidence of liver metastasis (table 3). Moreover, at this dose level, changes in body/organ weights or organ toxicities were not observed and a summary of median and range of tumor volumes and weights, incidence of liver metastasis and comparison with the effects of gemcitabine are presented in Table 3.

TABLE 3
Treatment of orthotopically implanted human pancreatic
L3.6p1 cancer cells by tolfenamic acid and gemcitabine.

| | | Pancreatic Tumor | | | | | | |
|---|---|---|---|---|---|---|---|---|
| Tumor | | Tumor | | Tumor | | Incidence | Body | |
| | Inci- | Volume (mm³) | | Weight (g) | | Liver | Weight (g) | |
| Group^{a} | dence^{b} | Median | Range | Median | Range | metastasis | Median | Range |
| Saline | 10/10 | 3587.9 | 985.7-8662.5 | 1.3 | 0.8-1.5 | 5/10 | 24 | 21-24 |
| Gemcitabine | 10/10 | 1186.8 | 606.4-1441.2 | 0.8 | 0.5-1.1 | 5/10 | 24 | 19-29 |
| (50 mg/kg) | | | | | | | | |
| Tolfenamic acid | 10/10 | 1732.8 | 984.7-2076.9 | 1.2 | 0.8-1.3 | 1/10 | 25 | 19-27 |
| (25 mg/kg) | | | | | | | | |
| Tolfenamic acid | 10/10 | 348.8^{c} | 128.3-840.5 | 0.4^{c} | 0.1-0.9 | 1/10 | 24 | 24-27 |
| (50 mg/kg) | | | | | | | | |

^{a}L3.6p1 human pancreatic cancer cells (1 × 10⁶) were injected into the pancreas of nude mice. Seven days later, different groups of mice were treated with bi-weekly intraperitoneal injections of gemcitabine (50 mg/kg), thrice weekly oral tolfenamic acid (25 mg/kg) or (50 mg/kg) or saline (control). All mice were killed on day 35.
^{b}Number of positive mice/number of mice injected.
^{c}p < 0.005 as compared to controls.

The results also indicated that at comparable doses tolfenamic acid was more effective than gemcitabine as a tumor growth inhibitor whereas the latter compound did not affect the incidence of liver metastasis at the dose used herein. The pancreatic tumors from the orthotropic model were also examined and the levels of Sp1, Sp3, Sp4 and VEGF proteins in tolfenamic acid vs DMSO treated animals were quantitated. Tolfenamic acid treatment significantly decreased expression of Sp1, Sp3, Sp4 and VEGF in pancreatic tumors (FIG. 8C) which paralleled the same responses observed in pancreatic cancer cells treated with this compound (FIGS. 1 and 4).

Immunostaining for VEGF in tumor sections from control, gemcitabine (50 mg/kg) and tolfenamic acid (25 and 50 mg/kg) treated animals also showed relatively high staining in tumors from control and gemcitabine-treated animals, but showed a decreased staining in tumors from mice treated with tolfenamic acid (FIG. 8D). In parallel studies, staining with CD31 to determine microvessel density showed decreased staining in tumors from mice treated with tolfenamic acid compared to tumors from vehicle (corn oil) or gemcitabine-treated animals (FIG. 8E). These results demonstrated that tolfenamic acid exhibited antitumorigenic and antiangiogenic activities in pancreatic cancer cells and tumors in vivo through degradation of Sp proteins which led to decreased VEGF expression and indicated that the diphenyl/diphenylamine carboxylic acid subclass of nonsteroidal antiinflammatory drugs were promising drugs for treatment of cancers such as pancreatic, breast, prostate, colon, bladder and ovarian cancers.

The following references were cited herein:

Abdelrahim et al., 2004, Cancer Res 64:6740-6749.

1. Abdelrahim et al., 2005, Mol Pharmacol 68:317-329.
2. Baker et al., 2002, Cancer Res 62:1996-2003.
3. Bruns et al., 1999, Neoplasia 1:50-62.
4. Dubois et al., 1998, FASEB J 12:1063-1073.
5. Shi et al., 2001, Cancer Res 61:4143-4154.
6. Masferrer et al., 2000, Cancer Res 60:1306-1311.
7. Wei et al., 2004, Cancer Res 62:2030-2038.
8. Bouwman P et al. Regulation of the activity of Sp1-related transcription factors. Mol Cell Endocrinol (2002) 195, 27-38
9. Black A R et al. J Cell Physiol 2001, 188
10. Safe S et al. Prog Nucleic Acid Res Mol Biol (2004) 77, 1-36
11. Shi Q et al. Cancer Res (2001) 61, 4143-4154
12. Wang L et al. Clin Cancer Res (2003) 9, 6371-6380
13. Yao J C et al. Clin Cancer Res (2004) 10, 4109-4117
14. Zannetti A et al. Cancer Res (2000) 60, 1546-1551
15. Chiefari E et al. BMC Cancer (2002) 2, 35-36
16. Hosoi Y et al. Int J Oncol (2004) 25, 461-468
17. Abdelrahim M et al. Cancer Res (2004) 64, 6740-6749
18. Wei D et al. Cancer Res (2004) 64, 2030-2038
19. Abdelrahim M et al. Mol Pharmcol (2005) 68,317-329
20. Baker C H et al. Cancer Res (2002) 62, 1996-2003
21. Bruns C J et al. Neoplasia (1999) 1, 50-62
22. Abdelrahim M et al. J Biol Chem (2005) 280,16508-16513
23. Abdelrahim M et al. Carcinogenesis (September 2005) (submitted)
24. Su K et al. J. Biol Chem (1999) 274, 15194-15202
25. Rock K et al. Cell (1994) 78, 761-771
26. Omura S J et al. Antibiot (Tokyo) (1991) 44, 113-118
27. Dick L R, J Biol Chem (1996) 271, 7273-7276
28. Sapetschnig A et al. J Biol Chem (2004) 279, 42095-42105
29. Johnson E S and Gupta A A. Cell (2001) 106, 735-744
30. Tatham M H et al. J Biol Chem (2001) 276, 35368-35374
31. Pichler A et al. Cell (2002) 108, 109-120
32. Rodriguez M S et al. J Biol Chem (2001) 276, 12654-12659
33. Sapetschnig A et al. EMBO J (2002) 21, 5206-5215
34. Bernier-Villamor V et al. Cell (2002) 108, 345-356
35. Bruan H et al. Nucleic Acid Res (2001) 29, 4994-5000
36. Black A R et al. J Cell Physiol (2001) 188, 143-160
37. Hart G W. Annu Rev Biochem (1997) 66, 315-335
38. Jackson S P and Tjian R. Cell (1988) 55, 125-133
39. Jackson S P et al. Cell (1990), 63, 155-165
40. Chu S and Ferro T J. Gene (2005) 348, 1-11
41. Ryu H et al. Proc Natl Acd Sci USA (2003) 100, 4281-4286
42. Suzuki T et al. Genes Cell (2000) 5, 29-41
43. Huang W et al. J Biol Chem (2005) 280, 10047-10054
44. Braun H et al. Nucleic Acid Res (2001) 29, 4994-5000
45. Chou T Y et al. Proc Natl Acad Sci USA (1995) 92, 4417-4421
46. Kelly W G et al. J Biol Chem 268, 10416-10424
47. Reason A J et al. J Biol Chem (1992) 267, 16911-16921
48. Tarnawski A S and Jones M K. J Mol Med (2003) 81, 627-636
49. Taketo M M. J Natl Cancer Inst (1998) 90, 1529-1536
50. Taketo M M. J Natl Cancer Inst (1998) 90, 1609-1620
51. Gately S et al. Semin Oncol (2004) 31, 2-11.
52. Giovannucci E et al. N Engl J Med (1995) 333, 609-614
53. Martinez M E et al. Cancer Epidemiol Biomarker Prev (1995) 4, 703-707
54. Pelge II et al. Dig Dis Sci (1996) 41, 1319-1326
55. Steinbach G et al. N Engl J Med (2000) 342, 1946-1952
56. Labayle D et al. Gastroenterology (1991) 101, 635-639
57. Nugent K P et al. Br J Surg (1993) 80, 1618-1619
58. Chiu C H et al. Cancer Res (1997) 57, 4267-4273
59. Boolbol S K et al. Cancer Res (1996) 56, 2556-2560
60. Pereir M A et al. Carcinogenesis (1994) 15, 1049-1054
61. Reddy S, Cancer Res (2000) 60, 293-297
62. Jacoby R F et al. Cancer Res (2000) 60, 5040-5044
63. Thun M et al. J Natl Caner Inst (2002) 94, 252-256
64. Thun M et al. Cancer Res (1993) 53, 1322-1327
65. Egan K M et al. J Natl Cancer Inst (1996) 88, 988-993
66. Harris R E et al. Oncol Rep (1999) 6, 71-73
67. Sharp C R et al. Br J Cancer (2000) 83, 112-120
68. Khuder S A et al. Br J Cancer (2001) 84, 1188-1192
69. Norrish A E et al. Int J Cancer (1998) 77, 511-555
70. Cramer D W et al. Lancet (1998) 351, 104-107
71. Rodriguez C et al. Lancet (1998) 352, 1354-1355
72. Tavani A et al. Ann Oncol (2000) 11, 1171-11173
73. Jacob E J et al. J Natl Cancer Inst (2005) 97, 975-980
74. Jacobs E J et al. Cancer Epidemiol Biomarkers Prev (2005) 14, 261-264
75. Lindblad M et al. Cancer Epid Biomarkers Prev (2005) 14, 444-450
76. Schernhammer E S et al. J Natl Cancer Inst (2004) 96, 22-28
77. Jacobe E J et al. J Natl Cancer Inst (2004) 96, 524-528
78. Anderson K E et al. J Natl Cancer Inst (2002) 94, 1168-1171
79. Menezes R J et al. BMC Public Health (2002) 2, 18-19

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A method of inducing degradation of one or more Sp transcription factors, comprising:
contacting a cancer cell with a non-steroidal anti-inflammatory drug or a substituted diphenylamine or diphenylamine carboxylic acid derivative, thereby inducing degradation of one or more Sp transcription factors.

2. The method of claim 1, wherein said degradation induces the expression of p27 such that proliferation of the cancer cell is inhibited.

3. The method of claim 1, wherein said degradation decreases the expression of VEGF such that angiogenesis, tumor metastasis or combination thereof in a tumor comprising said cancer cell is inhibited.

4. The method of claim 1, wherein the nonsteroidal antiinflammatory drug is a diphenyl/diphenylamine carboxylic acid.

5. The method of claim 4, wherein the diphenyl/diphenylamine carboxylic acid is tolfenamic acid, diclofenac sodium, or diflunisal.

6. The method of claim 1, wherein the Sp transcription factor is a Sp1 protein, a Sp3 protein, a Sp4 protein or a combination thereof.

7. The method of claim 1, wherein the cancer cell is a pancreatic cancer cell, a breast cancer cell, a prostate cancer cell, a colon cancer cell, a bladder cancer cell or an ovarian cancer cell.

8. A method of treating cancer in an individual, comprising:
administering a pharmacologically effective amount of diphenyl/diphenylamine carboxylic acid to the individual, thereby treating the cancer in the individual.

9. The method of claim 8, further comprising:
administering a different chemotherapeutic drug.

10. The method of claim 9, wherein the chemotherapeutic drug is administered concurrently or sequentially with the compound.

11. The method of claim 8, wherein the diphenyl/diphenylamine carboxylic acid inhibits tumor growth, angiogenesis and metastasis in the individual.

12. The method of claim 8, wherein the diphenyl/diphenylamine carboxylic acid is tolfenamic acid, diclofenac sodium, or diflunisal.

13. The method of claim 8, wherein the individual is diagnosed with pancreatic cancer, breast cancer, prostate cancer, colon cancer, bladder cancer or ovarian cancer.

14. A method of treating pancreatic cancer in an individual, comprising:
administering pharmacologically effective amount of tolfenamic acid, wherein the tolfenamic acid inhibits proliferation, angiogenesis and metastasis of the pancreatic cancer, thereby treating the pancreatic cancer in the individual.

15. The method of claim 14, further comprising:
administering a different chemotherapeutic drug.

16. The method of claim 15, wherein the chemotherapeutic drug is administered concurrently or sequentially with the tolfenamic acid.

17. A method of reducing toxicity of a cancer therapy in an individual in need thereof, comprising:
administering to the individual a diphenyl/diphenylamine carboxylic acid and another chemotherapeutic drug, wherein the dosage of the chemotherapeutic drug administered is lower than the dosage required when said chemotherapeutic drug is administered singly, thereby reducing the toxicity of the cancer therapy in the individual.

18. The method of claim 17, wherein the chemotherapeutic drug is administered concurrently or sequentially with the diphenyl/diphenylamine carboxylic acid.

19. The method of claim 18, wherein the diphenyl/diphenylamine carboxylic acid is tolfenamic acid, diclofenac sodium, or diflunisal.

20. The method of claim 18, wherein the cancer is pancreatic cancer, breast cancer, prostate cancer, colon cancer, bladder cancer or ovarian cancer.

21. A method of treating cancer in an individual, comprising:
administering a pharmacologically effective amount of diphenyl/diphenylamine carboxylic acid; and
a siRNA specific for one or more Sp transcription factors, to the individual, thereby treating the cancer in the individual.

24. The method of claim 21, wherein said treatment inhibits tumor growth, angiogenesis and/or metastasis in the individual.

25. The method of claim 21, wherein the diphenyl/diphenylamine carboxylic acid is tolfenamic acid, diclofenac sodium, or diflunisal.

26. The method of claim 21, wherein the individual is diagnosed with pancreatic cancer, breast cancer, prostate cancer, colon cancer, bladder cancer or ovarian cancer.

27. The method of claim 21, wherein the Sp transcription factor is a Sp1 protein, a Sp3 protein, a Sp4 protein or a combination thereof.

28. A method of inhibiting the angiogenic response of a tumor in an individual comprising:
administering a pharmacologically effective amount of diphenyl/diphenylamine carboxylic acid; and
a siRNA specific for one or more Sp transcription factors, to the individual, thereby inhibiting the angiogenic response of the tumor in said individual.

29. The method of claim 28, wherein said method decreases the expression of VEGFR1, such that angiogenic response of the tumor is inhibited.

30. The method of claim 28, wherein said treatment inhibits tumor growth and metastasis in the individual

31. The method of claim 28, wherein the diphenyl/diphenylamine carboxylic acid is tolfenamic acid, diclofenac sodium, or diflunisal.

32. The method of claim 28, wherein the individual has a pancreatic cancer, breast, prostate, colon, bladder or ovarian tumor.

33. A method of inhibiting tumor metastasis in an individual comprising:
administering a pharmacologically effective amount of diphenyl/diphenylamine carboxylic acid; and
a siRNA specific for one or more Sp transcription factors, to the individual, thereby inhibiting tumor metastasis in said individual.

34. The method of claim 33, wherein said method decreases the expression of VEGFR1, such that tumor metastasis is inhibited.

35. The method of claim 33, wherein the diphenyl/diphenylamine carboxylic acid is tolfenamic acid, diclofenac sodium, or diflunisal.

36. The method of claim 33, wherein the individual has a pancreatic cancer, breast, prostate, colon, bladder or ovarian tumor.


SEQUENCE LISTING (13 sequences)

SEQ ID NO: 1 - DNA, 21 bp, artificial sequence
  primer_binding: VEFG forward primer
ccatgaactttctgctgtctt

SEQ ID NO: 2 - DNA, 20 bp, artificial sequence
  primer_binding: VEFG reverse primer
atcgcatcaggggcacacag

SEQ ID NO: 3 - DNA, 20 bp, artificial sequence
  primer_binding: GAPDH forward primer
aatcccatcaccatcttcca

SEQ ID NO: 4 - DNA, 20 bp, artificial sequence
  primer_binding: GAPDH reverse primer
gtcatcatatttggcaggtt

SEQ ID NO: 5 - DNA, 20 bp, artificial sequence
  primer_binding: VEGFR1 forward primer
tgggacagtagaaagggctt

SEQ ID NO: 6 - DNA, 21 bp, artificial sequence
  primer_binding: VEGFR1 reverse primer
ggtccactccttacacgacaa

SEQ ID NO: 7 - DNA, 20 bp, artificial sequence
  primer_binding: GAPDH forward primer
aatcccatcaccatcttcca

SEQ ID NO: 8 - DNA, 20 bp, artificial sequence
  primer_binding: GAPDH reverse primer
gtcatcatatttggcaggtt

SEQ ID NO: 9 - DNA, 31 bp, artificial sequence
  primer_binding: plasmid pGAL4-Sp3B sense primer
tccgtcgacgcaacagcgtttctgcagctcc

SEQ ID NO: 10 - DNA, 33 bp, artificial sequence
  primer_binding: plasmid pGAL4-Sp3B antisense primer
tatctagaatcagccttgaattgggtgcacctg

SEQ ID NO: 11 - DNA, 32 bp, artificial sequence
  primer_binding: plasmid pGAL4-Sp3CD sense primer
tccggatccgcctgccgttggctatagcaaat

SEQ ID NO: 12 - DNA, 35 bp, artificial sequence
  primer_binding: plasmid pGAL4-Sp3CD antisense primer
gtatgtcgacatcagaagccattgccactgatatt

SEQ ID NO: 13 - DNA, 32 bp, artificial sequence; identical to SEQ ID NO: 11
  primer_binding: plasmid pGAL4-Sp3D sense primer